(19) United States
(12) Patent Application Publication
(10) Pub. No.: US 2013/0130311 A1
(43) Pub. Date: May 23, 2013

(54) METHODS AND SYSTEMS FOR ASSESSING CLONALITY OF CELL CULTURES

(71) Applicants: Ehud Y. Shapiro, Nataf (IL); Tuval Ben-Yehezkel, Rehovot (IL)

(72) Inventors: Ehud Y. Shapiro, Nataf (IL); Tuval Ben-Yehezkel, Rehovot (IL)

(73) Assignee: YEDA rESEARCH AND DEVELOPMENT CO., LTD., Rehovot (IL)

(21) Appl. No.: 13/812,530

(22) PCT Filed: Jul. 27, 2011
(86) PCT No.: PCT/IL11/00607

§ 371 Date: Jan. 28, 2013

(30) Foreign Application Priority Data
Jul. 27, 2010 (US) 61367880
Aug. 10, 2010 (US) 61372106
Oct. 6, 2010 (US) 61390232

(51) Int. Cl. C12Q 1/02

(52) U.S. Cl. CPC C12Q 1/02
USPC 435/39; 435/34; 435/29; 435/252.3; 435/471

(57) ABSTRACT

Methods of determining clonality of a cell culture are provided. Also provided are systems employing the above methods in high throughput sample screening.

### FIELD AND BACKGROUND OF THE INVENTION

The present invention, in some embodiments thereof, relates to methods and systems for assessing clonality of cell cultures.

Transformation followed by monoclonal culture of bacteria (i.e. cloning) is arguably the most widely used procedure in biological research, constituting a necessary step in most research and development efforts of molecular and cellular biology. At the turn of the 19^{th} century two methods for obtaining pure (monoclonal) culture of bacteria were established. The first was based on limiting dilution of bacteria in a liquid growth medium and the second was based on plating bacteria onto solid medium in Petri dishes. Since the advent of proper transformation methods for E. coli in the 1970's methods for obtaining pure bacterial culture have been widely used in order to isolate transformed clones. Nevertheless, biologists have largely neglected the original dilution-based method and instead routinely plate and pick colonies one-by-one from solid medium in Petri dishes.

Modern biological research attempts to take advantage of high throughput approaches. Indeed, conducting research that requires high throughput cloning using Petri dish technology is potentially highly rewarding^{3,4,5} but is seldom undertaken due to the cumbersome manual effort it entails. The ongoing revolution in high-throughput technologies (which generate significant data on multiple levels of functionality) is increasingly pervading almost every domain of biological research, further spurring the interest and strive for high-throughput. This, in turn, has made traditional cloning practice a bottleneck in many cases and relieving this bottleneck has become a subject of ongoing research. Unfortunately, even the most modern attempts at automating cloning fail to relieve the truly labor intensive and time consuming steps^{6,7}. Additionally, alternative solutions to eliminate manual labor, such as automated colony picking robots⁸ and in specific cases even a shift to cell-free in-vitro cloning^{9,10} have not enabled a widespread use of high throughput cloning.

### SUMMARY OF THE INVENTION

According to an aspect of some embodiments of the present invention there is provided a method of diagnosing an infection in a subject, the method comprising:

(a) culturing a fluid sample of the subject under conditions that allow a pathogen in the sample to propagate in a liquid culture, wherein a propagation of the pathogen comprises an initial lag phase and a subsequent growth phase;

(b) analyzing a rate of increase of an optical density of the liquid culture during the growth phase; and

(c) determining the time required to reach the growth phase;

wherein a combination of the rate of increase above a predetermined level and the time required to reach the growth phase below a predetermined level is indicative of the infection in the subject.

According to an aspect of some embodiments of the present invention there is provided a method of determining colony forming units (CFU) of a pathogen, the method comprising:

(a) culturing a fluid sample under conditions that allow a pathogen in the sample to propagate in a liquid culture, wherein a propagation of the pathogen comprises an initial lag phase and a subsequent growth phase;

(b) analyzing a rate of increase of an optical density of the liquid culture during the growth phase; and

(c) determining the time required to reach the growth phase;

wherein a combination of the rate of increase and the time required to reach the growth phase correlates to the CFU.

According to an aspect of some embodiments of the present invention there is provided a method of diagnosing an infection in a subject, the method comprising:

(a) serially diluting a cell culture comprising a fluid sample of the subject; and

(b) determining in the serial dilutions time to a predetermined OD, the time required for each of the serial dilutions to gain a predetermined OD being indicative of the infection.

According to an aspect of some embodiments of the present invention there is provided a method of determining clonality of a cell culture comprising:

providing a cell culture which comprises cells expressing a plurality of distinct reporter polypeptides, each of the plurality of distinct reporter polypeptides being expressed by different cells of the cell culture,

wherein the plurality of distinct reporter polypeptides are selected:

(i) having distinctive signals;

(ii) generating a distinctive signal when co-expressed in a culture, the distinctive signal being distinguishable from the distinctive signals; determining clonality of the cell culture based on expression of the plurality of distinct reporter polypeptides, wherein a presence of the distinctive signal is indicative of a non-clonal culture and wherein an absence of distinctive signal is indicative of a clonal culture.

According to an aspect of some embodiments of the present invention there is provided an isolated population of cells comprising competent cells the competent cells expressing an exogenous recombinant polynucleotide encoding a reporter polypeptide.

According to some embodiments of the invention, the recombinant polynucleotide is not a translational fusion.

According to some embodiments of the invention, the plurality of distinct reporter polypeptides are fluorescent polypeptides.

According to some embodiments of the invention, the cell culture is a prokaryotic culture.

According to some embodiments of the invention, the prokaryotic culture comprises pathogenic cells.

According to some embodiments of the invention, the cell culture is a eukaryotic culture.

According to some embodiments of the invention, the method further comprises diluting the cell culture prior to determining clonality.

According to some embodiments of the invention, the method further comprises determining a level of the distinctive signal in a reference culture.

According to some embodiments of the invention, the reference culture is a polyclonal culture.

According to some embodiments of the invention, the reference culture is a monoclonal culture.

According to some embodiments of the invention, at least one of the plurality of distinct reporter polypeptides is not a translational fusion.

According to some embodiments of the invention, the culture comprises competent cells.

According to some embodiments of the invention, the cell culture is a liquid culture.

According to some embodiments of the invention, the cell culture comprises cells transformed with a polynucleotide of interest.

According to some embodiments of the invention, the method further comprising sequencing the polynucleotide of interest in the clonal culture.

According to an aspect of some embodiments of the present invention there is provided a method of determining colony forming units (CFU), the method comprising:

serially diluting a cell culture; and

determining in the serial dilutions time to a predetermined OD, the time required for each of the serial dilutions to gain a predetermined OD correlates to its respective colony count.

According to an aspect of some embodiments of the present invention there is provided a method of determining clonality of a cell culture, the method comprising:

culturing the cell culture; and

monitoring time to a predetermined OD, wherein a time to OD of a predetermined value is indicative of a monoclonal cell culture.

According to some embodiments of the invention, the method further comprising generating a calibration curve of time to OD as a function of CFU.

According to some embodiments of the invention, the monitoring comprises real-time monitoring.

According to some embodiments of the invention, the method further comprising diluting the cell-culture to a single cell culture following the monitoring.

According to some embodiments of the invention, the method further comprising testing synchronization of the cell culture.

According to some embodiments of the invention, the method further comprising synchronizing the cell culture by diluting the cell culture.

According to some embodiments of the invention, the cell culture comprises transformed cells.

According to an aspect of some embodiments of the present invention there is provided a method of determining clonality of a cell culture, comprising analyzing the as described above.

According to an aspect of some embodiments of the present invention there is provided a method of cloning comprising:

transforming competent cells with a polynucleotide of interest, so as to obtain transformed cells;

identifying a clone expressing the polynucleotide of interest according to the method above;

sequencing the clone so as to identify the polynucleotide of interest.

According to some embodiments of the invention, the method is automated.

According to some embodiments of the invention, the method is robotics-assisted.

According to an aspect of some embodiments of the present invention there is provided a method of synchronizing a plurality of cell cultures, the method comprising:

simultaneously monitoring OD of each of the plurality of cell cultures, wherein an OD distribution range which exceeds a predetermined value is indicative of non-synchronized cell cultures; and

diluting the cell cultures of the plurality of cell cultures exhibiting an OD value which exceeds a predetermined value so as to minimize the OD distribution range and synchronize the plurality of cell cultures.

According to an aspect of some embodiments of the present invention there is provided a system for determining clonality of a cell culture, the system comprising a processing unit, the processing unit executing a software application configured for monitoring time to a predetermined OD, wherein a time to OD of a predetermined range is indicative of a monoclonal cell culture.

According to an aspect of some embodiments of the present invention there is provided a system for determining clonality of a cell culture the system comprising a processing unit, the processing unit executing a software application configured for determining clonality of the cell culture based on expression of a plurality of distinct reporter polypeptides in the cell culture, each of the plurality of distinct reporter polypeptides being expressed by different cells of the cell culture,

wherein the plurality of distinct reporter polypeptides are selected:

(i) having distinctive signals;

(ii) generating a distinctive signal when co-expressed in a culture, the distinctive signal being distinguishable from the distinctive signals.

According to some embodiments of the invention, the system further comprises a programmable laboratory robot for obtaining the time to OD or the expression of the plurality of distinct reporter polypeptides.

According to some embodiments of the invention, the programmable laboratory robot comprises a machine selected from the group consisting of a PCR machine, an electrophoresis apparatus, a signal detection apparatus, a CCD camera, a sequencing device and an actuator.

According to some embodiments of the invention, the infection is a bacterial infection.

According to some embodiments of the invention, the infection is a fungal infection.

According to some embodiments of the invention, the fluid sample is selected from the group consisting of urine, cerebrospinal fluid, semen, plasma and blood.

According to some embodiments of the invention, the method further comprises confirming a result of the diagnosis.

According to some embodiments of the invention, the method further comprises informing the subject of a result of the diagnosis.

According to some embodiments of the invention, the liquid culture comprises culture medium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show the process of accurately performing end-to-end automated cloning in liquid. FIG. 1A—Transformed bacterial culture in liquid growth medium are diluted until optical density (OD) measurement is equal to the blank measurement of the medium. FIG. 1B—The diluted bacteria (a) are then cultured with real-time OD monitoring until they gain a predefined OD value, from which the CFU is also inferred. FIG. 1C—The monitored culture (b) is immediately diluted by a predefined factor with liquid growth medium. FIG. 1D-aliquots of the diluted culture (c) are plated in 384 multi-well plates to robustly produce a predefined amount of addressable bacterial clones per plate. Verified clones can be used for any downstream application.

FIGS. 2A-B shows that CFU correlates to time-to-OD. FIG. 2A—Several transformed bacteria were cultured with constant OD monitoring (blue, green, purple) in order to time their growth to a predetermined OD value. Identical transformation samples were plated on LB agar Petri dishes and in multi-well plates in order to determine their colony forming unit (CFU) value. FIG. 2B—The time required for each of the cultures (a. top—blue, green, purple etc.) to gain a predefined OD value robustly correlates to its respective colony count (A., Petri dish and multi-well colony counts). This establishes an accurate correlation between liquid CFU and time to OD and is used to rapidly determine the CFU value in the present method. Additionally, the comprehensive colony count performed for both plating in liquid and solid medium indicate that plating in liquid medium produces ˜100 times more colonies than plating onto solid medium.

FIGS. 3A-C shows the accuracy and dynamic range of the limiting dilution method. Three samples of transformed bacteria spanning a wide range of CFU values (3A—a, b and c) were diluted and cultured under constant OD monitoring until each gained exactly the same OD value. Upon reaching the predetermined OD each culture was serially diluted in LB (10³, 10⁴, 10⁵, 10⁶ and 10⁷). For each initial culture (a, b and c) sixteen wells from each of the five dilutions were plated, cultured overnight in multi-well plates and OD read (3B—a, b and c). In addition, sixteen negative control wells (liquid LB) were plated together with the dilutions of each initial culture (3B—bottom rows). Subsequent OD measurement of the plated dilutions displays a (positive or negative) growth pattern reflecting a digital pattern of either 0 or >=1 bacteria per well at plating (3B—a, b and c). More importantly, although transformations with widely varying CFU values were used (3A—a, b and c) the ratio of positive to negative wells ebbs in a similar manner among the three different cultures (3C—a, b and c) after the constant limiting dilution procedure. This indicates that the limiting dilution method has a wide dynamic range and enables the cloning of transformations with widely varying efficiencies (or CFU values) using the exact same procedure.

FIGS. 4A-D demonstrate clonal verification. FIGS. 4A-B. polyclonal (A) and monoclonal (B) fluorescent signatures. Each well of a 384 well plate post cloning is subjected to the appropriate fluorescence measurement of all four proteins. Wells of monoclonal origin (4B) display an emission signature only for one of the proteins. Polyclonal wells (4A) display emission signatures for more than one protein. C. Clonality verification using DNA sequencing of bar-coded DNA. Polyclonal cultures (top chromatogram) are easily distinguishable from monoclonal cultures (bottom chromatogram) due to insertions, deletions and substitutions. FIG. 4D. The probability for obtaining true (monoclonal) clones is maximized when cells are plated at an average concentration of one cell per well (blue plot). Nevertheless, at this concentration there is a considerable probability of wells being polyclonal (red plot, ˜25%). By using fluorescent detection (A-B) the probability of false positive (polyclonal) wells is reduced considerably (black plot) and the optimal concentration (one cell per well) can be used. Alternatively, a lower average number of cells per well can be used at the price of more negative (no growth) wells.

FIG. 5 is a diagram illustrating a system for assessing clonality of a cell culture according to some embodiments of the present invention.

FIG. 6 is a diagram illustrating an automated laboratory including a system for assessing clonality of a cell culture according to some embodiments of the present invention.

FIG. 7 is a graph showing transformations with different efficiencies are cultured and monitored for OD in real time and the time it takes them to reach a predetermined OD value is recorded. The CFU of the transformation is then inferred from this information.

FIG. 8 is a graph showing that identical triplet transformations (four shown here), which have the same CFU value, also exhibit an identical time-to-OD.

FIGS. 9A-G are graphs exemplifying that the number of positive wells (clones) is linearly correlated with the dilution factor used in the dilution for single cells. The first bar represents orange, the second bar represents cherry, the third bar represents citron, the fourth bar represents tangerine and the fifth bar represents CFP.

FIGS. 10A-E demonstrates fluorescent detection of monoclonality. Monoclonal signatures of all four fluorescent proteins are presented. Each of the four fluorescent clones was measured with the excitation and emission wavelengths of all four proteins. Each bar graph shows the measurement of each clone at all four excitation/emission wavelengths. The order from top to bottom is: Citrine, Tangerine, Cherry and Orange. The figures show fluorescence measurement and corresponding DNA sequence of exemplary positive and false positive clones. Note that monoclonal cultures show a perfect sequence while polyclonal cultures exhibit mutations (since they harbor more than one type of DNA sequence) which cause the sequencing chromatogram to be shifted and unreadable from the mutation position and on.

FIG. 11 is an image of automated colony PCR. The gel shows electrophoresis of 16 colony PCR's executed according to the optimized conditions. The fragments are at the correct size (768 bp) and were amenable to high quality sequencing.

FIGS. 12A-C are graphs illustrating turbidity rise in urine samples over time. FIG. 12A represents the turbidity rise <3 hours (3/44 samples). FIG. 12B represents turbidity rise >5 hours (36/44 samples). FIG. 12C represents turbidity rise 3-5 hours (5-44 samples).

### DESCRIPTION OF SPECIFIC EMBODIMENTS OF THE INVENTION

The present invention, in some embodiments thereof, relates methods and systems for assessing clonality of cell cultures.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cloning of bacteria was first introduced over a century ago and has since become one of the most useful procedures in biological research, perhaps paralleled in its ubiquity only by PCR and DNA sequencing. However, unlike PCR and sequencing, cloning has generally remained a manual, labor-intensive low-throughput procedure. In the age of high-throughput biological research, cloning has remained a bottleneck for most labs, largely unrelieved despite the advent of sophisticated and specialized equipment such as automated colony pickers and cell sorters.

The present inventors have devised a novel approach of computer-aided automated bacterial cloning in liquid medium that is based on the principles of limiting dilution, analog CFU inference and fluorescent clonal verification. The utility of the method for the ever-increasing number of high throughput research and diagnostic applications is demonstrated by employing it as a cloning platform for a DNA synthesis process.

Technological advances and the increasing requirements for high throughput cloning call for a reexamination of how monoclonal bacterial culture is obtained. The present inventors have realized that high throughput cloning can be effected by reverting to how cloning was first demonstrated over a century ago by Joseph Lister², using a dilution based technique. Dilution-based pure culture of bacteria has been largely neglected as a routine method. Nevertheless, relatively recent technological advancements (e.g., plate readers, liquid handling robots) enabled to develop a cloning method based on using liquid growth medium which is more suitable for automation using standard liquid handling robots. The method can also be executed in a non-automated manual setup. It enables the process of cloning, starting from untransformed bacteria and ending with sequenced plasmids, to be automated with high throughput. It is confined to standard multi-well plates that can be managed and analyzed in real time by in-house developed software and processed with high throughput using liquid handling robots and plate readers.

Terminology and general methods (other terms and methods are described elsewhere in the document).

As used herein the term “clone” refers to a group of genetically identical cells derived from a single cell by replication. A clone is also referred to herein as a monoclonal culture.

As used herein the term “clonality” refers to the condition of being a clone. Also referred to as being clonal, or relating to a clone.

As used herein the phrase “cell culture” refers to a culture in a proliferative phase.

The cell culture comprises cells. Cells may be eukaryotic (e.g., human, mammal, plant, yeast, insect) or prokaryotic (e.g., bacterial cell culture).

The culture may be a liquid culture.

According to a specific embodiment the cells are competent cells. Competence refers to the ability of a cell to take up extracellular DNA from its environment.

Methods of generating competent cells are well known in the art and further described hereinbelow.

According to specific embodiments the cells are transformed to express a polynucleotide-of-interest (expression at the RNA and optionally protein level).

Methods of transforming cells are well known in the art and further described hereinbelow.

Thus, according to an aspect of the invention there is provided a method of determining clonality of a cell culture comprising:

providing a cell culture which comprises cells expressing a plurality of distinct reporter polypeptides, each of the plurality of distinct reporter polypeptides being expressed by different cells of the cell culture,

wherein the plurality of distinct reporter polypeptides are selected:

(i) having distinctive signals;

(ii) generating a distinctive signal when co-expressed in a culture, the distinctive signal being distinguishable from the distinctive signals;

determining clonality of the cell culture based on expression of the plurality of distinct reporter polypeptides, wherein a presence of the distinctive signal is indicative of a non-clonal culture (polyclonal) and wherein an absence of distinctive signal is indicative of a clonal culture (monoclonal).

As mentioned the cell culture according to this aspect expresses a plurality of distinct reporter polypeptides.

As used herein the term “plurality” refers to at least two, e.g., 2, 3, 4, 5, 6, 7, 8, 9, 10 or even more.

The phrase “reporter polypeptide” as used herein, refers to a polypeptide which can be detected in a cell. Preferably, the reporter polypeptide of this aspect of the present invention can be directly detected in the cell (no need for a detectable moiety with an affinity to the reporter) by exerting a detectable signal which can be viewed in living cells (e.g., using a fluorescent microscope). Non-limiting examples of reporter polypeptides include fluorescent reporter polypeptides, (e.g. those comprising an autofluorescent activity), chemiluminescent reporter polypeptides and phosphorescent reporter polypeptides. Examples of fluorescent polypeptides include those belonging to the green fluorescent protein family, including but not limited to the green fluorescent protein, the yellow fluorescent protein, the cyan fluorescent protein and the red fluorescent protein as well as their enhanced derivatives. See also the Examples section which follows, for additional examples. The reporter polypeptides of this aspect of the invention elicit distinctive (i.e., distinguishable from each other) signals.

Thus, fluorescent reporter polypeptides for example may be selected such that each emits light of a distinguishable wavelength and therefore color when excited by light.

The fluorescent reporter polypeptides are further selected such that when co-expressed in culture generate a distinctive signal (i.e., distinguishable). The distinctive signal may be a signature, i.e., a collection (i.e., sum) of the distinctive signals or a de-novo signal such as a result of Fluorescence Resonance Energy Transfer (FRET) (see e.g., Hui-Wang et al. Nat. Methods 2008 5:401-403, incorporated herein by reference).

According to a specific embodiment, polynucleotide sequences encoding the reporter polypeptides are integrated into the genome of the cells, so as to provide for a stable culture. However, episomal expression is also contemplated by the present teachings.

Following in Tables 1 and 2 is a non-limiting list of fluorescent proteins which may be used in accordance with the present teachings.

TABLE 1

| | | Excitation | | | Photo- | | Oligo- |
|---|---|---|---|---|---|---|---|
| Class | protein | (nm) | Emission | Brightness | stability | pKa | merization |
| Far-red | mPlum | 590 | 649 | 4.1 | 53 | <4.5 | Monomer |
| Red | mCherry | 587 | 610 | 16 | 96 | <4.5 | Monomer |
| | tdTomato | 554 | 581 | 95 | 98 | 4.7 | Tandem |
| | | | | | | | dimer |
| | mStrawberry | 574 | 596 | 26 | 15 | <4.5 | Monomer |
| | J-Red | 584 | 610 | 8.8 | 13 | 5 | dimer |
| | DsRed- | 556 | 586 | 3.5 | 16 | 4.5 | Monomer |
| | monomer | | | | | | |
| Orange | mOrange | 548 | 562 | 49 | 9.0 | 6.5 | Monomer |
| | mKO | 548 | 559 | 31 | 122 | 5.0 | Monomer |
| Yellow- | mCitrine | 516 | 529 | 59 | 49 | 5.7 | Monomer |
| green | | | | | | | |
| | Venus | 515 | 528 | 53 | 15 | 6.0 | Weak |
| | | | | | | | dimer |
| | Ypet | 517 | 530 | 80 | 49 | 5.6 | Weak |
| | | | | | | | dimer |
| | EYFP | 514 | 527 | 51 | 60 | 6.9 | Weak |
| | | | | | | | dimer |
| Green | Emerald | 487 | 509 | 39 | 0.69 | 6.0 | Weak |
| | | | | | | | dimer |
| | EGFP | 488 | 507 | 34 | 174 | 6.0 | Weak |
| | | | | | | | dimer |
| Cyan | CyPet | 435 | 477 | 18 | 59 | 5.0 | Weak |
| | | | | | | | dimer |
| | mCFPm | 433 | 475 | 13 | 64 | 4.7 | monomer |
| | Cerulean | 433 | 475 | 27 | 36 | 4.7 | Weak |
| | | | | | | | dimer |
| UV- | T-Saphhire | 390 | 511 | 26 | 25 | 4.9 | Weak |
| excitable | | | | | | | dimer |
| green | | | | | | | |

TABLE 2

| | Fluorescent | | |
|---|---|---|---|
| | protein | Excitation | Emission |
| Multiple labeling | Cerulean or CyPet | 425/20 | 484/40 |
| | mCitrine of YPet | 495/10 | 525/20 |
| | mOrange of mKO | 545/10 | 575/25 |
| | mCherry | 585/20 | 675/130 |
| | mPlum | 585/20 | 675/130 |

As mentioned, determining the clonality of the cell culture is based on the expression of the plurality of distinct reporter polypeptides. Signal detection can be done by a fluorescent plate reader. Preferably the detected signal is also imaged such as by using a CCD camera.

In order to determine the level of the signal and remove background signal (signal-to-noise), a control reference sample is typically included in the test. The reference sample may be a polyclonal sample in which the distinctive signals are expressed to elicit the distinctive signal, a monoclonal sample in which the distinctive signal is not detected (e.g., in a fluorescent plate reader), or a sample which comprises cells of the same species and competence level which do not express any reporter polypeptide (i.e., autofluorescent). The unique fluorescence emission signature from each culture is then recorded for future reference.

According to a specific embodiment, at least one of the reporter polypeptides is not expressed as a translational fusion (e.g., in specific embodiments all the fusion polypeptides are not expressed as translational fusions).

Anywhere during the protocol, the culture may be diluted (e.g., serial dilution) to improve the chances of obtaining single cell cultures and hence clones.

The process of determining clonality thus may be iterative. That is, the culture is diluted, signal is detected, if a distinctive signal is detected the sample may be further diluted and a presence of distinctive signal is detected until diminished.

Bacterial colony enumeration is an essential tool for many widely used assays and accurately determining the number of colony forming units (CFU) is a basic feature of any bacterial cloning method. Determining the number of discrete colonies formed, or CFU, can often be an exhaustive task, especially if the number of colonies and/or plates is very large as is the case in high throughput research and diagnostics. Additionally, counting and/or processing colonies on Petri dishes can often be problematic due to too high or too low CFU values and often requires plating a range of different dilutions to achieve the optimal value.

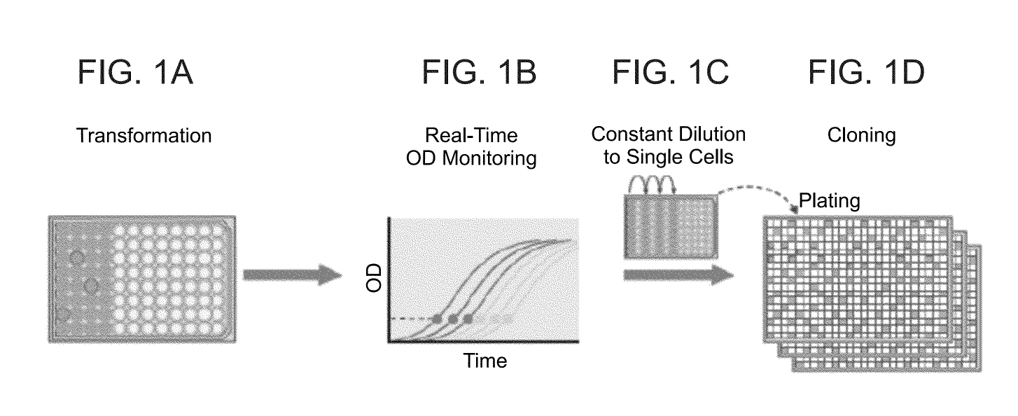

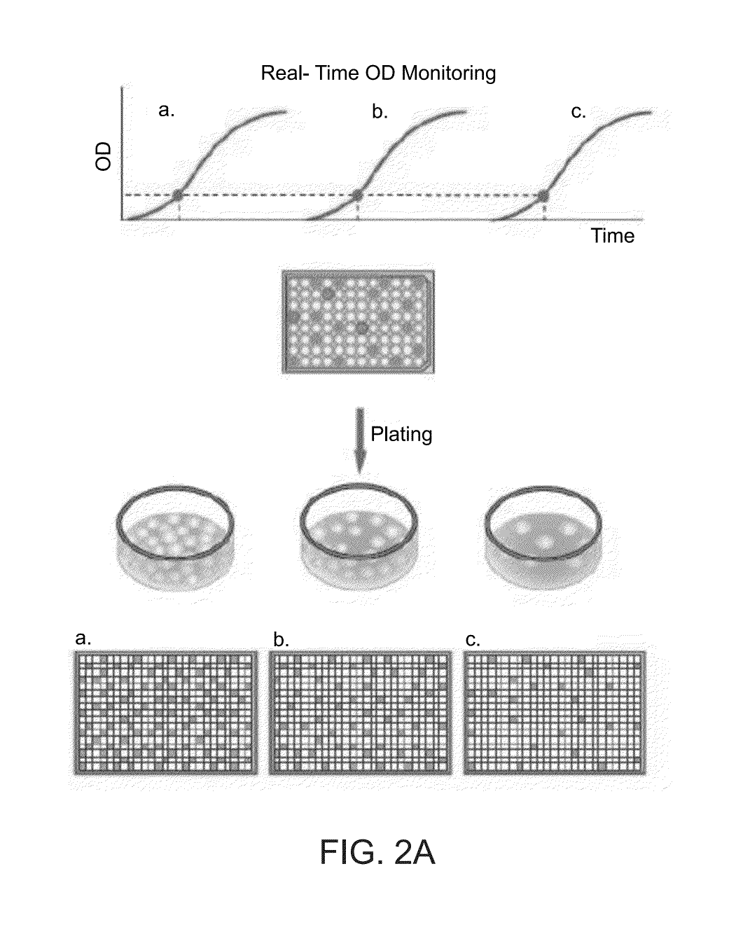

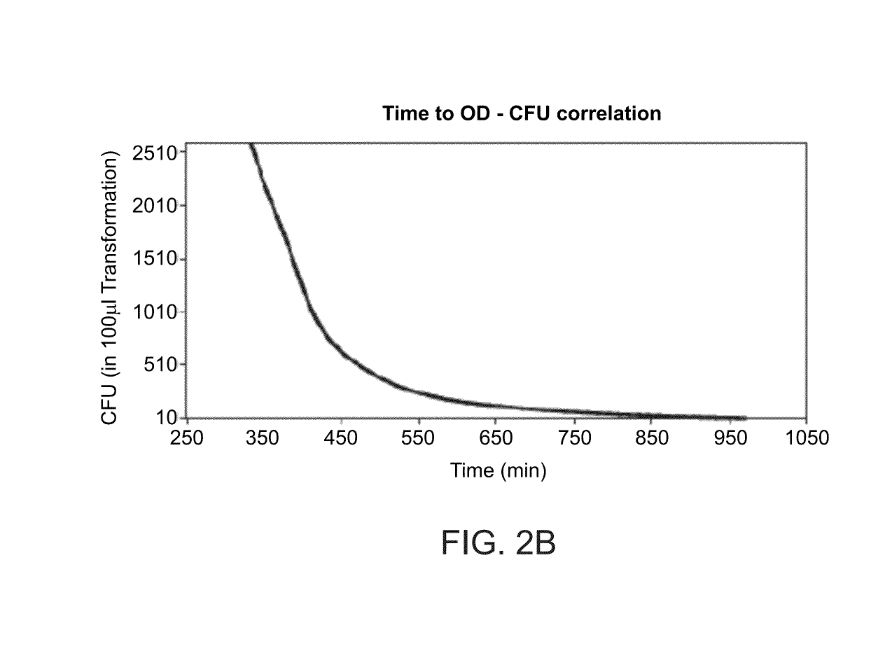

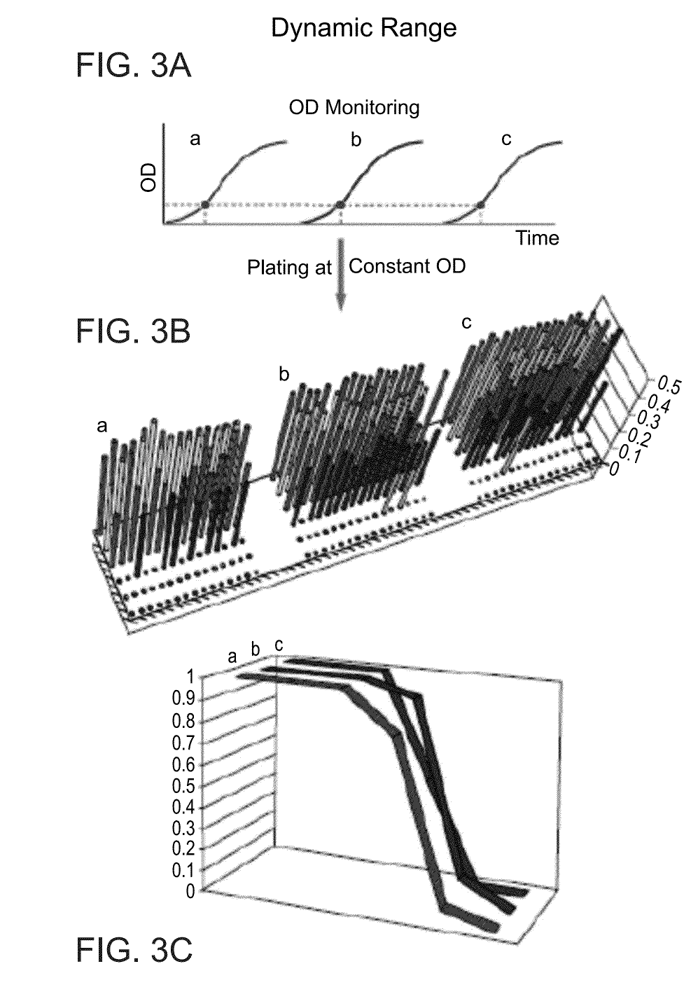

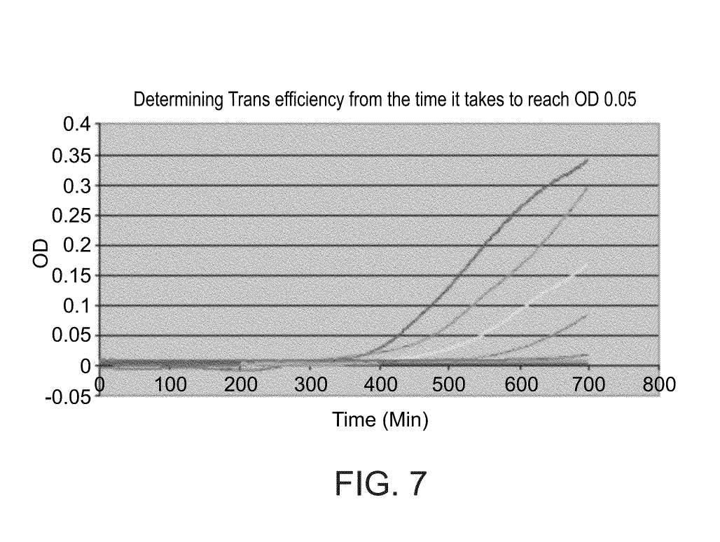

The present inventors have further observed that CFU accurately correlates to the time it takes a transformed culture to gain a predetermined OD value (FIG. 2a). The analysis is performed on data from the initial growth to OD phase (FIG. 1) that is required in any case. Thus the present methodology uses a continuous analog signal (time to OD) that can be obtained with high-throughput using an automated plate reader scan. The correlation between time-to-OD and CFU was found to be valid across the practical range of CFU values (See FIG. 2a and FIG. 7). For example, accurate CFU values of 96/384 separate transformations can be determined within minutes by timing each wells growth to the predetermined OD.

According to an aspect of the invention, there is provided a method of determining colony forming units (CFU) of a pathogen, the method comprising:

serially diluting a cell culture comprising the pathogen; and

determining in said serial dilutions time to a predetermined OD, the time required for each of said serial dilutions to gain a predetermined OD correlates to its respective colony count.

The present inventors have devised a further method for determining CFU of a pathogen. The method compises:

(a) culturing a fluid sample under conditions that allow the pathogen in the sample to propagate in a liquid culture, wherein a propagation of the pathogen comprises an initial lag phase and a subsequent growth phase;

(b) analyzing a rate of increase of an OD of the liquid culture during the growth phase; and

(c) determining the time required to reach the growth phase;

wherein a combination of said rate of increase and said time required to reach said growth phase correlates to the CFU.

Exemplary fluid samples which may be analyzed according to this aspect of the present invention include, but are not limited to biological samples derived from animals (e.g. humans), water samples, food and beverage samples.

The pathogen may be a bacteria or a fungus.

The bacteria may be gram positive or gram negative bacteria.

The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abscessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Sarcina lutea, Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

The term “Gram-negative bacteria” as used herein refer to bacteria characterized by the presence of a double membrane surrounding each bacterial cell. Representative Gram-negative bacteria include Acinetobacter calcoaceticus, Acinetobacter baumannii, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella pneumoniae, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Shigella sonnei, Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

The term “fungi” as used herein refers to the heterotrophic organisms characterized by the presence of a chitinous cell wall, and in the majority of species, filamentous growth as multicellular hyphae. Examples of fungi for which the CFU may be determined include, but are not limited to Candida albicans, Saccharomyces cerevisiae, Candida glabrata, Candida parapsilosis and Candida dubliniensis.

It will be appreciated that the rate of growth of a pathogen can be divided into four phases—an initial lag phase (or recovery phase), a growth or exponential phase (also referred to herein as the log phase), a stationery phase and a death phase. By graphically plotting the course of growth of the pathogen (e.g. by analyzing the OD of the culture), these four phases may be clearly distinguished one from the other.

The present inventors have found that the rate of increase of the growth phase (i.e. the gradient of the slope) together with the time taken to reach the growth phase (i.e. the length of time of the lag phase) may be used to determine the CFU of the pathogen.

One way of determining the time at which the growth phase begins is to analyze the signal to noise ratio of the recordings. the time at which a culture (e.g. urine culture) passes a threshold signal; average noise ratio is indicative of the time at which the growth phase begins. Thus, for example a signal:average noise ratio is determined throughout the experiment, and the time at which the growth phase begins may be estimated to be the point at which the signal:average noise ratio is at least twice (e.g. at least three times, at least 4 times, at least 5 times or more) the signal:average noise ratio.

The weight of each individual component (i.e. the gradient of the slope and the length of time of the lag phase) may be determined by routine experimentation using known positive and negative controls.

According to a further aspect of the invention there is provided a method of determining clonality of a cell culture, the method comprising: culturing said cell culture; and monitoring time to a predetermined optical density (OD), wherein a time to OD of a predetermined range is indicative of a monoclonal (i.e., clonal) cell culture.

As used herein “optical density” represents cell density e.g., in cells/ml. Cell density is usually determined at an OD of 600 nm.

Monitoring OD is preferably effected by real-time monitoring such as by using an automated spectrophotometer plate reader.

At times the growth rate of the culture should be determined. In this case a calibration curve is preferably generated, i.e., the suspension culture is serially diluted so as to obtain a series of samples such as with OD₆₀₀ of =2, 1, 0.8, 0.6, 0.4, 0.2 and 0.1.

For each OD the cell density in cells/mL is calculated. So for each OD, dilutions of 1 in 1×10̂7, 1×10̂6 and 1×10̂5, are made and then plated (1 mL) of each onto suitable plates. Colonies are grown and then the number of colonies formed on the dilution gives the most appropriate number multiplied up by the dilution factor to obtain the number of cells/mL in the original sample. These values can then be used to construct a calibration curve of OD vs cells/mL.

As mentioned, when the ratio of clonal/non-clonal cultures is too low the cultures may be diluted following said monitoring.

Likewise, synchronization of the culture should be examined. Unsynchronized cultures should be synchronized such as by dilution. Other limiting factors may be controlled to synchronize the cultures. For example, glucoamylase which controls growth-limiting release of glucose maintains synchronized growth of the cultures to similar cell densities.

Accordingly, the present invention also envisages a method of synchronizing a plurality of cell cultures, the method comprising:

simultaneously monitoring OD of each of said plurality of cell cultures, wherein an OD distribution range which exceeds a predetermined value is indicative of non-synchronized cell cultures; and

diluting said cell cultures of said plurality of cell cultures exhibiting an OD value which exceeds a predetermined value so as to minimize said OD distribution range and synchronize said plurality of cell cultures.

The process may be iterative as further dilutions may be needed.

Any of the above methods can be used individually or the combination may be practiced.

Any of the methods for determining clonality are preferably effected in microwell dishes which can be analysed in fluorescent/optic density plate readers (e.g., 6, 12, 24, 48, 96, 384 wells etc.).

Once a potential clone has been identified its localization in the array of samples is marked and a sample from the potential clone is retrieved and subject to DNA purification, PCR, sequencing or a combination of same.

Any of the above described methods can be practiced using automatic equipment such as robotics-assisted.

Methods of assessing clonality of cultures can be implemented in cloning, research and diagnostics. Preferably throughout any of these procedures the working environment is kept closed and sterile.

According to specific embodiments, the methods are implemented in diagnostics where CFU may be indicative of pathogenic (e.g. bacterial) infection. Amongst the advantages of the presently described methods for diagnosis of an infection, is the speed with which a positive diagnosis may be made (typically less than 6 hours and even less than four hours).

According to a particular embodiment, CFU above a predetermined threshold may be indicative of pathogenic infection. Thus, for example, a CFU greater than 10,000/ml may be indicative of a pathogenic infection. According to another embodiment, a CFU greater than 50,000/ml is indicative of a pathogenic infection. According to still another embodiment, a CFU greater than 100,000/ml is indicative of a pathogenic infection. It will be appreciated that the CFU threshold which is used to determine whether a subject has an infection may change according to the bodily fluid being analyzed.

According to another embodiment, the rate of increase of the growth phase (i.e. the gradient of the slope) in combination with the time taken to reach the growth phase (i.e. the length of time of the lag phase) may be used to determine the CFU of the pathogen.

Thus, if the rate of increase of the growth phase is above a predetermined level and the time taken to reach the growth phase is below a predetermined level this is indicative that the subject has an infection. If, in contrast the rate of increase of the growth phase is below a predetermined level and the time taken to reach the growth phase is above a predetermined level this is indicative that the subject is free of infection.

Biological fluids which may be analyzed include, but are not limited to urine, cerebrospinal fluid, vaginal discharge, semen, plasma and blood.

According to a particular embodiment, the biological fluid is urine and the diagnosis is for a bacterial urine infection.

It will be appreciated that for biological fluids that are not conducive to bacterial growth (e.g. urine), culture medium may be added to the sample in order to reduce the time of the lag phase of growth. The ratio of culture medium to biological fluid may be determined by routine experimentation.

Establishing diagnostics may be done using Gold-standard methods.

Following qualification that a sample is infected or not, antibiotics may be added to the culture in order to establish the identity of the pathogen and/or in order to select a particular antibiotic that may be useful for treating the infection. Thus, reduction in OD following addition of an antibiotic known to be effective against a particular bacteria (or class of bacterium) aids in the establishment of pathogen identity and accordingly selection of a therapeutic treatment. Conversely, if the OD of the culture does not change following addition of an antibiotic known to be effective against a particular bacteria (or class of bacterium), one may rule out that the infection is due to that particular bacteria, and accordingly rule out the use of that antibiotic as a therapeutic for treating the infection.

Other embodiments of the present invention provide for a method of cloning comprising:

transforming competent cells with a polynucleotide of interest, so as to obtain transformed cells;

identifying a clone expressing the polynucleotide of interest according to the method above;

sequencing the clone so as to identify the polynucleotide of interest.

It will be appreciated that the present invention relates also to competent cells per se. Thus, an aspect of the invention contemplates competent cells comprising an exogenous recombinant polynucleotide encoding a reporter polypeptide (such as described hereinbelow).

Methods of generating competent bacteria are well known in the art. Basically, there are two main methods for preparation of competent bacterial cells for transformation, the calcium chloride and the electroporation method. For the electrocompetent cell preparation see (Rakesh C. Sharma and Robert T. Schimke, “Preparation of Electro-competent E. coli Using Salt-free Growth Medium”, Biotechniques 20, 42-44 (1996), which is hereby incorporated by reference. For the calcium chloride method, a glycerol cell culture stock of the respective E. coli strain is thawed and added to 50 ml of liquid media. This culture then is preincubated at 37° C. for 1 hour, transferred to an incubator-shaker, and is incubated further for 2-3 hours. The cells are pelleted by centrifugation, resuspended in calcium chloride solution, and incubated in an ice-water bath. After another centrifugation step, the resulting cell pellet again is resuspended in calcium chloride to yield the final competent cell suspension. Competent cells are stored at 4 degC, for up to several days.

Methods of transforming/transfecting/infecting cells are well known in the art.

Transfection/transformation/transduction is the process of deliberately introducing nucleic acids into cells. Transfection is used notably for non-viral methods in eukaryotic cells. It may also refer to other methods and cell types, although other terms are preferred: “transformation” is more often used to describe non-viral DNA transfer in bacteria, non-animal eukaryotic cells and plant cells—a distinctive sense of transformation refers to spontaneous genetic modifications (mutations to cancerous cells (Carcinogenesis), or under stress (UV irradiation)). “Transduction” is often used to describe virus-mediated DNA transfer.

According to the present invention the transfection relates to the introduction of genetic material. Transfection of animal cells typically involves opening transient pores or “holes” in the cell membrane, to allow the uptake of material. Transfection can be carried out using calcium phosphate, by electroporation, or by mixing a cationic lipid with the material to produce liposomes, which fuse with the cell membrane and deposit their cargo inside. Any method of transfection/transformation/infection is contemplated by the present teachings. Some are summarized infra.

Chemical-Based Transfection

Chemical-based transfection can be divided into several kinds: cyclodextrin, polymers, liposomes, or nanoparticles (with or without chemical or viral functionalization. See below). The calcium phosphate employs HEPES-buffered saline solution (HeBS) containing phosphate ions which is combined with a calcium chloride solution containing the DNA to be transfected. When the two are combined, a fine precipitate of the positively charged calcium and the negatively charged phosphate will form, binding the DNA to be transfected on its surface. The suspension of the precipitate is then added to the cells to be transfected (usually a cell culture grown in a monolayer). By a process not entirely understood, the cells take up some of the precipitate, and with it, the DNA. Other methods use highly branched organic compounds, so-called dendrimers, to bind the DNA and get it into the cell. A very efficient method is the inclusion of the DNA to be transfected in liposomes, i.e. small, membrane-bounded bodies that are in some ways similar to the structure of a cell and can actually fuse with the cell membrane, releasing the DNA into the cell. For eukaryotic cells, transfection is better achieved using cationic lipids (or mixtures), because the cells are more sensitive DOPA, Lipofectamine™ and UptiFectin™ may be used.

Another method is the use of cationic polymers such as DEAE-dextran or polyethylenimine. The negatively charged DNA binds to the polycation and the complex is taken up by the cell via endocytosis. Popular agents of this type are the Fugene or LT-1, and JetPEI.

Non Chemical Methods

Electroporation is a popular method, although requiring an instrument and affecting the viability of many cell types, that also creates micro-sized holes transiently in the plasma membrane of cells under an electric discharge.

Similarly, transfection applying sonic forces to cells, referred as Sono-poration.

Optical transfection is a method where a tiny (˜1 μm diameter) hole is transiently generated in the plasma membrane of a cell using a highly focussed laser. This technique was first described in 1984 by Tsukakoshi et al., who used a frequency tripled Nd:YAG to generate stable and transient transfection of normal rat kidney cells^{[13]}. In this technique, one cell at a time is treated, making it particularly useful for single cell analysis.

Gene electrotransfer is a technique that enables transfer of genetic material into prokaryotic or eukaryotic cells. It is based on a physical method named electroporation, where transient increase in the permeability of cell membrane is achieved when submitted to short and intense electric pulses.

Particle-Based Methods

A direct approach to transfection is the gene gun, where the DNA is coupled to a nanoparticle of an inert solid (commonly gold) which is then “shot” directly into the target cell's nucleus.

Magnetofection, or Magnet assisted transfection is a transfection method, which uses magnetic force to deliver DNA into target cells. Nucleic acids are first associated with magnetic nanoparticles. Then, application of magnetic force drives the nucleic acid particle complexes towards and into the target cells, where the cargo is released.

Impalefection is carried out by impaling cells by elongated nanostructures such as carbon nanofibers or silicon nanowires which have been functionalized with plasmid DNA.

Viral Methods

DNA can also be introduced into cells using viruses as a carrier. In such cases, the technique is called viral transduction, and the cells are said to be transduced.

Other methods of transfection include nucleofection, heat shock.

The gene of interest may be stably or transiently expressed in the cell.

Usually, selection is enforced in order to identify those clones that express the gene of interest and to select out those cells not expressing the gene of interest.

Methods of sequencing are well known in the art (manual and automated). Automated DNA-sequencing instruments (DNA sequencers) can sequence up to 384 DNA samples in a single batch (nm) in up to 24 runs a day. DNA sequencers carry out capillary electrophoresis for size separation, detection and recording of dye fluorescence, and data output as fluorescent peak trace chromatograms. Sequencing reactions by thermocycling, cleanup and re-suspension in a buffer solution before loading onto the sequencer are performed separately. A number of commercial and non-commercial software packages can trim low-quality DNA traces automatically. These programs score the quality of each peak and remove low-quality base peaks (generally located at the ends of the sequence). The accuracy of such algorithms is below visual examination by a human operator, but sufficient for automated processing of large sequence data sets.

Most sequencing approaches use an in vitro cloning step to amplify individual DNA molecules, because their molecular detection methods are not sensitive enough for single molecule sequencing. Emulsion PCR isolates individual DNA molecules along with primer-coated beads in aqueous droplets within an oil phase. Polymerase chain reaction (PCR) then coats each bead with clonal copies of the DNA molecule followed by immobilization for later sequencing. Emulsion PCR is used in the methods by Marguilis et al. (commercialized by 454 Life Sciences), Shendure and Porreca et al. (also known as “Polony sequencing”) and SOLiD sequencing, (developed by Agencourt, now Applied Biosystems). Another method for in vitro clonal amplification is bridge PCR, where fragments are amplified upon primers attached to a solid surface, used in the Illumina Genome Analyzer. The single-molecule method developed by Stephen Quake's laboratory (later commercialized by Helicos) is an exception: it uses bright fluorophores and laser excitation to detect pyrosequencing events from individual DNA molecules fixed to a surface, eliminating the need for molecular amplification.

As mentioned, the methods of the present invention may be automated, such as by using a system as described below.

Accordingly, the methods of the present invention are carried out using a dedicated computational system.

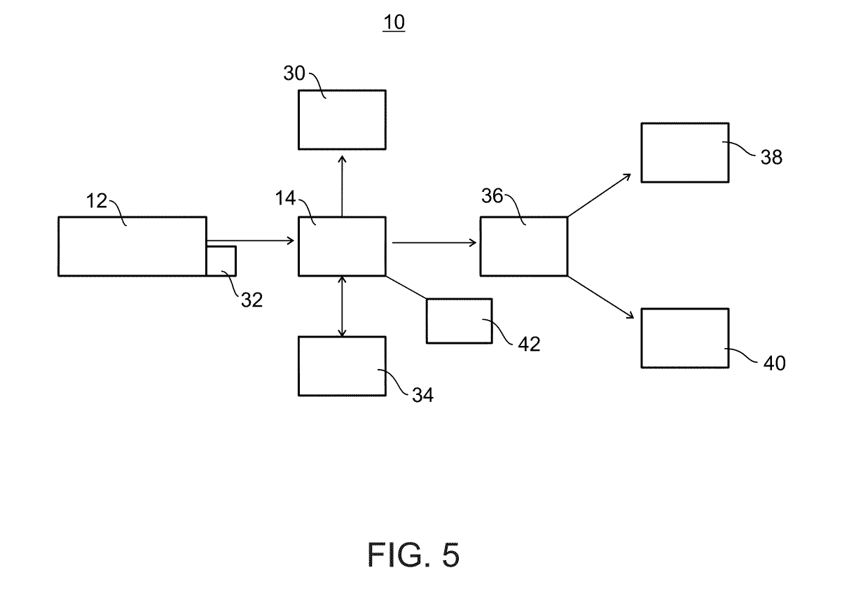

Thus, according to another aspect of the present invention and as illustrated in FIG. 5, there is provided a system for clonality of a cell culture which system is referred to hereinunder as system 10.

System 10 of this aspect of the present invention comprises a signal detection apparatus 12 (e.g., a spectrophotometer) being in functional communication with computing unit 14 for determining clonality of the cell culture as described hereinabove. The algorithm which executes the method receives input data from signal detection apparatus 12 and possibly CCD camera 32 connected to signal detection apparatus 12. The algorithm uses a set of rules such as described hereinabove and in the Examples section which follows, and determines whether a culture is monoclonal or polyclonal based on these sets of rules. One or more signal detection apparati (12) can be included in system 10. For example, a spectrophotometer plate reader can be included for determining OD and a fluorescent plate reader can be included for determining clonaility based on fluorescent signals as described above. Microplate fluprescent plate readers are available from Dynex Technologies, Perseptive Biosystems, Molecular Devices Corporation, Biotek, Tecan, Corona electric, PerkinElmer and the like. Preferably these plate readers are selected to handle a plurality of samples e.g., 384 samples.

Computing unit 14 and module 34 may be included in any computing platform 22 known in the art including but not limited to, a personal computer, a laptop, a work station, a mainframe and the like. Computing platform 22 can include display 30 for displaying for example a processed image of an examined culture, say a clonal culture is marked by plate number and coordinates thereof in the plate.

Computing unit 14 is in functional communication with coltroller 36 (e.g., chip). Controller 36 receives data from computing unit 14 and forwards it to either printer 18 or conveyer 40. Conveyer 40 is an actuator which may automatically move the culture plate, either for further signal detection, sample dilution, PCR and/or DNA sequencing etc.

System 10 preferably stores information generated thereby on a computer readable medium 42 such as a magnetic, optico-magnetic or optical disk.

Computing platform 22 may also include a user output interface 18 (e.g., a monitor, a printer) for providing clonal information to a user.

Any of the connections of system 10 (marked by arrows in FIG. 5) can be wired [e.g., a universal serial bus (USB) cable, a network connection wire] or wireless (e.g., by radiofrequency, wireless network connection).

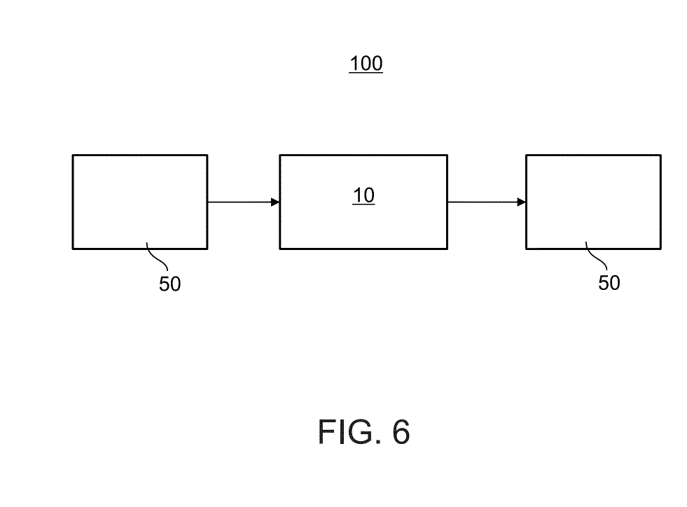

Alternatively, clonal data is generated by a programmable laboratory robot 100 (FIG. 6) which comprises a plurality of fluid handling robots 50 connected to system 10. Fluid handling robots 50 are configured for aliquoting a cell culture into microplate, incubating same in physiological degrees so as to allow culture propagation, transferring same to signal detection apparati and determining clonality as states above, amplifying a gene of inetrest from a clonal culture and sequencing in a partly or wholly automatic manner. Hence laboratory robot may comprise for example, a PCR machine, an electrophoresis apparatus, a sequencing device and actuator and an incubator. Laboratory robots can be commercially obtained such as from Life Science Automation wwwdotssiroboticsdotcom/Life-Sciences.html.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

All the automated procedures listed below were executed using the Tecan Freedom EVO® 200 System™ (Tecan, Mannedorf, Switzerland) with the relevant automated peripheral equipment. Real time analysis and control over the processes was executed using specialized in-house developed software. Robot control scripts using this in-house developed software for the automated procedures listed below can be found in Example 7, below.

Automated Tailing and Ligation

PCR fragments were ligated into the pGEM T easy Vector System1™ (Promega, Madison, Wis., U.S.A). The PCR products were A′ tailed at the 3′ using Hot-Start Taq polymerase (ABgene, Epsom, United Kingdom).

Ligation was performed in 10 μl using T4 DNA ligase (Promega) according to the manufacturer's specifications.

Automated Transformation

Transformations were performed using an automated procedure (See script) into Z-Competent E. coli (Zymo research, Orange, Calif., U.S.A) according to manufacturer's specifications.

Automated Inoculation

Cells were taken out of the plate reader-incubator at a predetermined OD value and diluted by a factor of 5*10⁶ with LB (See script). 30 ul inoculations were dispersed into 384 well plates with 40 ul LB to a total volume of 70 ul.

Automated Plasmid Purification

Clones were grown overnight in 1.3 ml LB and plasmids were extracted from them using the QuickClean 96 Well Plasmid Miniprep Kit (Genscript, Piscataway, N.J. U.S.A) according to manufacturer's specifications.

Automated PCR Purification

PCR reactions were purified using the Zymo research DNA Clean & Concentrator™-5 kit according to manufacturers specifications.

Automated PCR and Tailing

PCR reactions were performed using the Accusure™ hot start enzyme (Bioline, Taunton Mass., U.S.A) and tailing was executed using hot start Taq polymerase (ABgene) according to manufacturers specifications.

Automated Sequencing

Automated sequencing was performed using the BigDye® Terminator v3.1 Cycle Sequencing Kit (ABI, Carlsbad, Calif., U.S.A) according to manufacturers specifications and the sequencing reactions were purified with the 96 well Performa® DTR kit (EdgeBio, Gaithersburg, Md., U.S.A). Purified sequencing reactions were run on a 3730×1 96-capillary DNA Analyzer (ABI).

Competent Fluorescent Bacteria Protocol

Fluorescent E. coli expressing the Cherry, Tangerine, Orange and Citrine genes (AY678264—Cherry, AY678270—Tangerine, AY678265—Orange, 1HUY—Citrine) were made competent for transformation with a LiAc based procedure according to: Molecular Cloning: A Laboratory Manual, Chapterl, Third Edition (CSHL press).

Fluorescent Proteins

Plasmids from which the fluorescent proteins were expressed were of the Prset™ series developed at the Tsien lab ([Nature Biotechnology 22, 1567-1562 (2004)].

Automated Colony PCR

1 ml overnight cultures of E. coli JM109 in deep multi-well plates were pelleted, re-suspended in 250 ul DDW using 1450 rpm on a Tecan Te-Shake™ plate shaker, incubated at 95° C. for 10 minutes and diluted 1:10 in DDW. 5 μl aliquots of this dilution were used as template in PCR reactions.

Recursive Construction and Error Correction of the Synthetic Library Cloned

The core recursive construction and reconstruction (error-correction) step requires four basic enzymatic reactions: phosphorylation, elongation, PCR and Lambda exonucleation. They are described in the order of execution by the present protocol: Phosphorylation of all PCR primers used by the recursive construction protocol is performed beforehand simultaneously, according to the following protocol: 300 pmol of 5′ DNA termini in a 50 μl reaction containing 70 mM Tris-HCl, 10 mM MgCl₂, 7 mM dithiothreitol, pH 7.6 at 37° C., 1 mM ATP, 10 units T4 Polynucleotide Kinase (NEB, Ipswich, Mass., U.S.A). Incubation is at 37° C. for 30 min, inactivation 65° C. for 20 min.

Overlap Extension Elongation Between Two ssDNA Fragments:

1-5 pmol of 5′ DNA termini of each progenitor in a reaction containing 25 mM TAPS pH 9.3 at 25° C., 2 mM MgCl₂, 50 mM KCl, 1 mM β-mercaptoethanol 200 μM each of dNTP, 4 units Thermo-Start DNA Polymerase (ABgene). Thermal cycling program is as follows: Enzyme activation at 95° C. 15 min, slow annealing 0.1° C./sec from 95° C. to 62° C., elongation at 72° C. for 10 min.

PCR Amplification of the Above Elongation Product with Two Primers, One of Which is Phosphorylated:

1-0.1 fmol template, 10 pmol of each primer in a 25 μl reaction containing 25 mM TAPS pH 9.3 at 25° C., 2 mM MgCl₂, 50 mM KCl, 1 mM β-mercaptoethanol 200 μM each of dNTP, 1.9 units AccuSure DNA Polymerase (BioLINE). Thermal Cycler program is: Enzyme activation at 95° C. 10 min, Denaturation 95° C., Annealing at Tm of primers, Extention 72° C. 1.5 min per kb to be amplified 20 cycles.

Lambda Exonuclease Digestion of the Above PCR Product to Re-Generate ssDNA:

1-5 pmol of 5′ phosphorylated DNA termini in a reaction containing 25 mM TAPS pH 9.3 at 25° C., 2 mM MgCl₂, 50 mM KCl, 1 mM β-mercaptoethanol 5 mM 1,4-Dithiothreitol, 5 units Lambda Exonuclease (Epicentre, Madison, Wis., U.S.A). Thermal Cycler program is 37° C. 15 min, 42° C. 2 min, Enzyme inactivation at 70° C. 10 min.

### Example 2

### Time to OD

The following is a high level description of the cloning procedure, followed by specific, in-depth descriptions and discussion of the implemented methodologies

Description of the Method

Competent bacteria that do not require heat shock or recovery (Zymo research, Z competent) are transformed in 96-well plates with an automated procedure (See Methods). After transformation the cultures are automatically diluted into transparent 96 well plate with a selective liquid growth medium (LB-Amp) to a point that their OD value is identical to the blank growth medium (0.075±0.005 using 300 ul LB-Amp aliquots). These diluted transformations are then cultured at 37° C. in a plate reader (Tecan Infinite® 200 PRO series, Mannedorf, Switzerland) with real-time OD monitoring. The growth of each transformation it automatically timed from its initial blank value 0.075±0.005 OD to a predefined value of 0.1±0.005 OD. If the OD of all transformed cultures is not synchronized (due to different CFU) then they are synchronized using an automated (and potentially iterative) dilution-based synchronization procedure prior to single cell inoculation. T his enables to determine the colony forming units, as shown in FIGS. 2A-B and described in detail in ‘Analog CFU inference’ below. Once all transformations are at 0.1±0.005 OD, the transformations are diluted by a factor of 5*10⁵ (this is a mere example and other dilution factors can be used), from which single cells are inoculated into 384 well plates using 30 μl aliquots. In principle, other OD values which are still within the range of linear correlation to cell density can also be used, albeit with a different dilution factor to single cells. Once grown overnight the 384 well plates display a predefined ratio of positive (growth) to negative (no growth) wells of three. Positive and negative well positions within 384 well plates are determined by a plate reader OD scan. Verification of clonality is accomplished by a combination of an appropriately low positive/negative well ratio (concentrations under 1 cell per well) and fluorescent bacteria that report whether they are monoclonal, as described in ‘Verification of monoclonality using fluorescent bacteria’. The monoclonal positive wells (true clones) can then be used for downstream applications.

Analog CFU Inference

Bacterial colony enumeration is an essential tool for many widely used assays and determining the number of colony forming units (CFU) is a basic feature of bacterial cloning. In certain cases, such as high throughput research and diagnostics, determining the CFU can be an exhaustive task. Additionally, enumerating and/or processing colonies from Petri dishes can be problematic due to too high or too low CFU values^{11,12} and often requires plating a range of different dilutions to achieve the optimal value.

Specific embodiments of the invention relate to inferring CFU based on the observation that CFU correlates to the time it takes a transformed culture to gain a predetermined OD value (See FIG. 2a). For example, a culture starting from 10³ transformed cells (CFU equal to 10³) will reach an OD of 0.1 exactly one doubling time slower than a culture that started from 2*10³ cells (CFU equal to 2*10³). The correlation between time-to-OD and CFU was found to be valid across the practical range of CFU values (See FIG. 2a). In the context of the present cloning method this analysis is performed on data from the initial growth to OD phase (FIG. 1) that is required in any case in order to accurately dilute to less than single cells. This time-to-OD property of a culture can be obtained with high-throughput using a plate reader scan. For example, accurate CFU values of 96/384 separate transformations can determined automatically by timing each wells growth to the predetermined OD. In special cases in which the culture has a significantly different growth rate than that of standard E. coli strains used in most labs or produce some protein or material that significantly alters their growth rate a calibration experiment that determines their growth rate should be executed and analyzed to produce standard curves such as that presented in FIG. 2a.

Plating in Liquid Offers a 100-Fold Increase in Transformation Efficiency

The efficiency of the process, as measured in CFU, is increased due to cloning into liquid instead of solid medium. Of note, comparison shows that although identical samples of transformed cells were inoculated, cloning to liquid repeatedly exhibits a CFU value ˜100-fold higher compared to cloning onto solid LB in Petri dishes (See FIG. 2b). This 100-fold increase in CFU may be useful when DNA libraries are larger than the practical CFU value due to the large size of the library and/or inefficiencies of preceding steps.

Verification of Clones

Statistically, the low ratio of positive to negative wells chosen (3 negative wells for each positive well) favors the monoclonality of positive wells. Nevertheless, possible factors, such as the adhesion of several bacteria or contamination by other bacteria, can't be excluded as generators of false positive (polyclonal) cultures. DNA sequencing was used in order to determine the frequency of such false positive instances to test the integrity of the method, as follows:

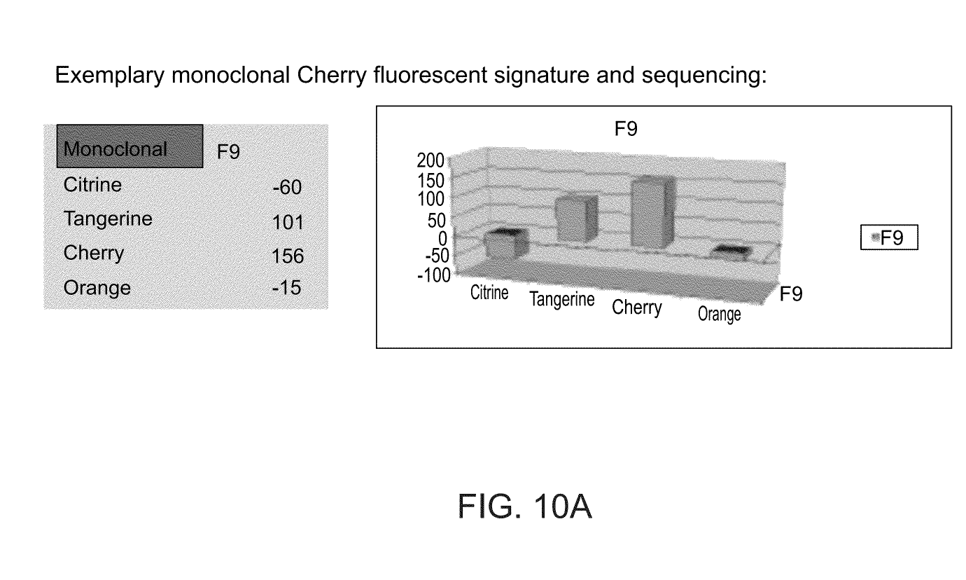

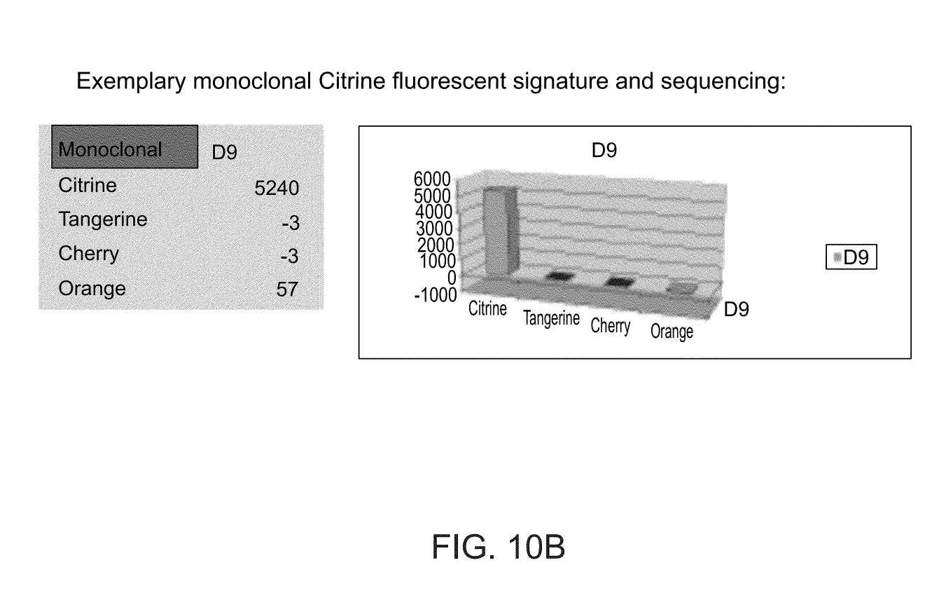

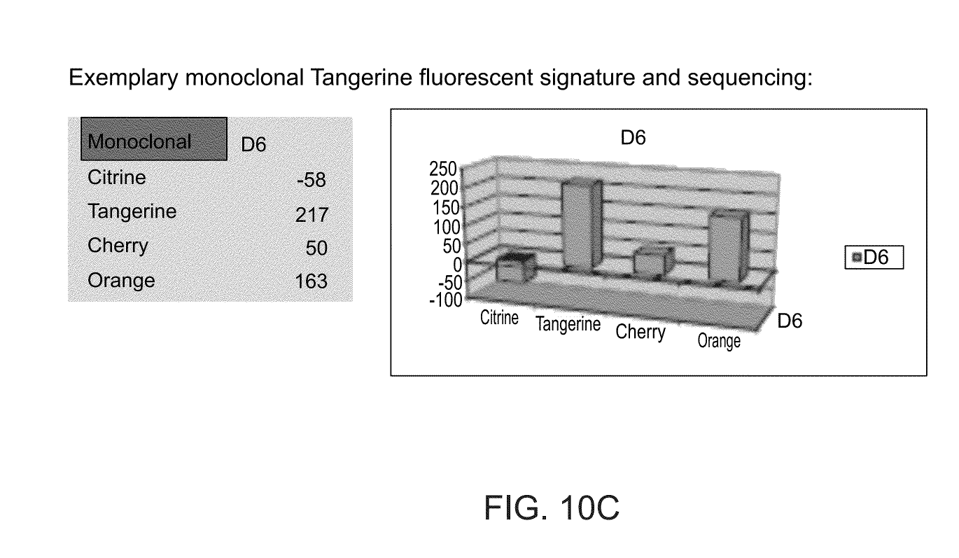

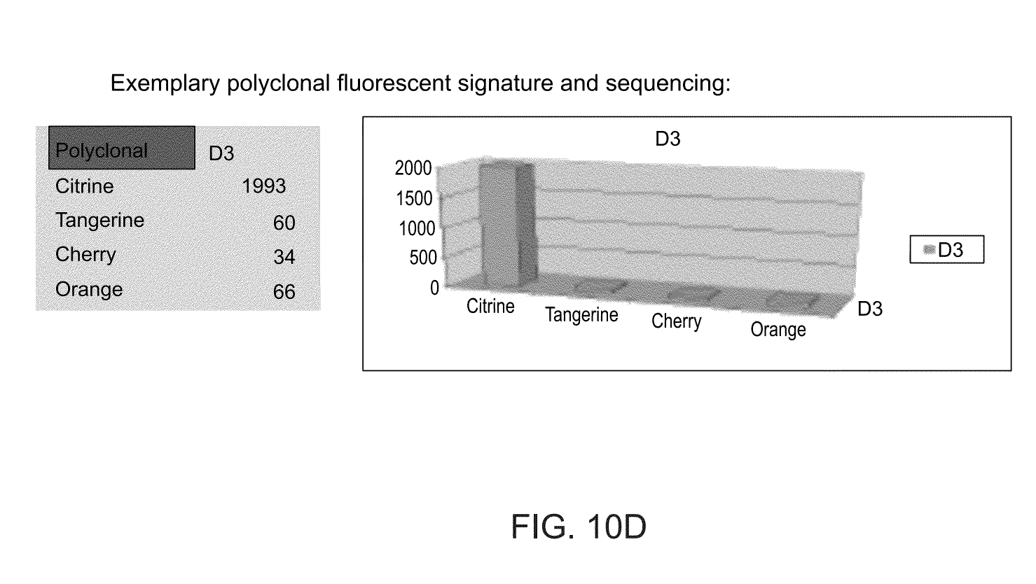

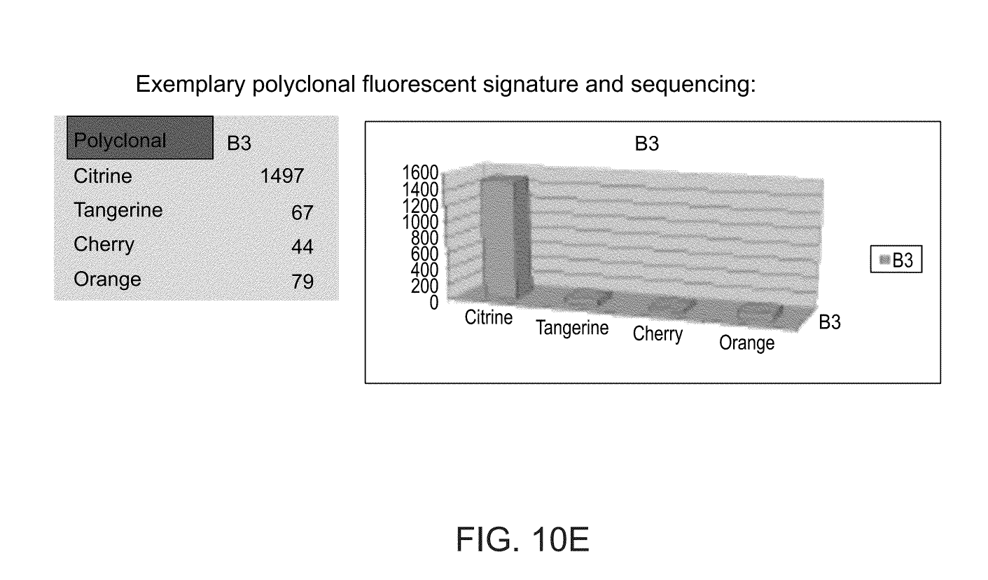

The cloned DNA used in all cloning experiments was a synthetic DNA library 768 nt long, with a high frequency of mutations. A comprehensive sequencing analysis of this library showed that the molecules of the library have 4-5 mutations per DNA molecule on average and that mutation positioning is random¹³ along the 768 bases cloned. As a result, the molecules of this library are in effect bar-coded, since each had a unique pattern of mutations that was practically impossible to clone twice. Monoclonal and polyclonal cultures of this cloned library are easily distinguishable using DNA sequencing since polyclonal cultures always harbor more than a single plasmid sequence due to the high error-rate of the library (See FIGS. 10A-E). Sequencing results after cloning were obtained by manually plating positive wells from our cloning procedure onto Petri dishes. Each positive well was plated onto a separate Petri dish and several colonies from each Petri dish were manually picked and sequenced. Sequencing analysis shows that colonies picked from the same Petri dish (i.e. inoculated with cells from a single positive well) reproducibly propagated plasmids with the exact same pattern of mutation. Conversely, colonies from different Petri dishes (i.e. colonies inoculated from different positive wells) reproducibly propagated plasmids exhibiting a completely different pattern of mutation, which never repeated itself. The fact that cells from the same positive well always exhibit the same sequence combined with the fact that cells from different positive wells never exhibit the same sequence attests to the monoclonality of positive wells. These results exclude phenomena such as cell aggregates or contamination as generators of false-positive results. Therefore, false positive clones are not produced at any significant rate which compromises the effectiveness of the method when a low ratio of positive to negative wells is used.

Cloning Ratio

The maximal fraction of monoclonal cultures is obtained when the average number of viable cells/aliquot is one (i.e., 1 viable cell/aliquot). Nevertheless, the cost and effort of sequencing false positive (i.e. polyclonal) colonies outweighs that of plating more negative wells. Therefore, it is reasonable to aim for a low ratio of positive/negative wells. A ratio of 1/3. is therefore chosen. After the initial growth to OD 0.01 is reached, the bacteria are diluted by a factor of 5×10⁵ to a ratio of 1/3 negative wells for each positive well on average using 30 ul inoculation aliquots into 384-well plates with 40 ul in each well, totaling 70 ul in each well. This ratio ensures a high probability of clonal amplification in positive wells.

### Example 3

### Verification of Monoclonality Using Fluorescent Bacteria

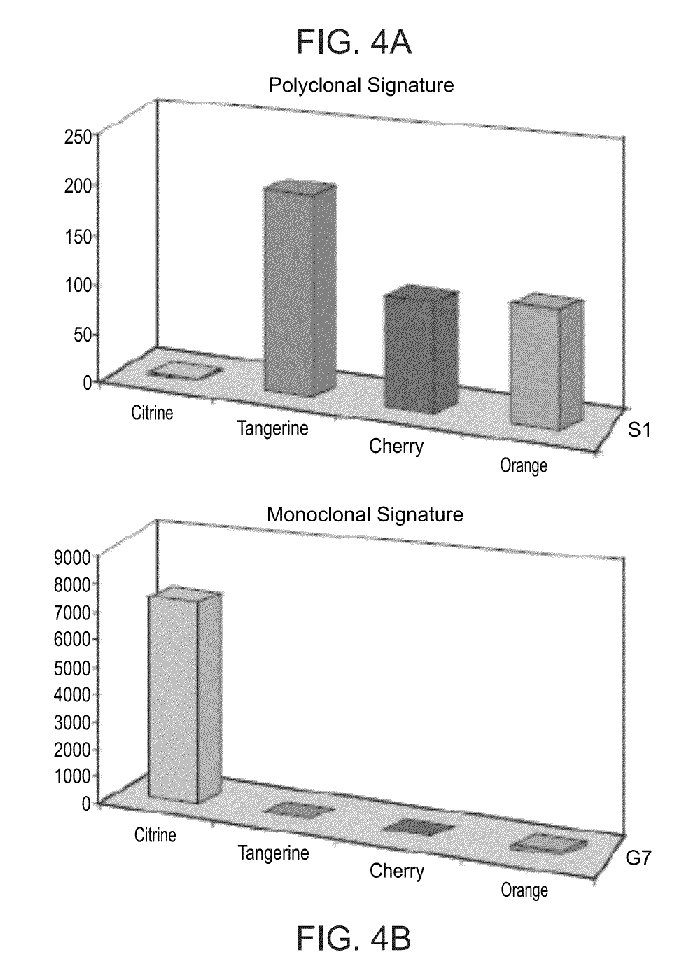

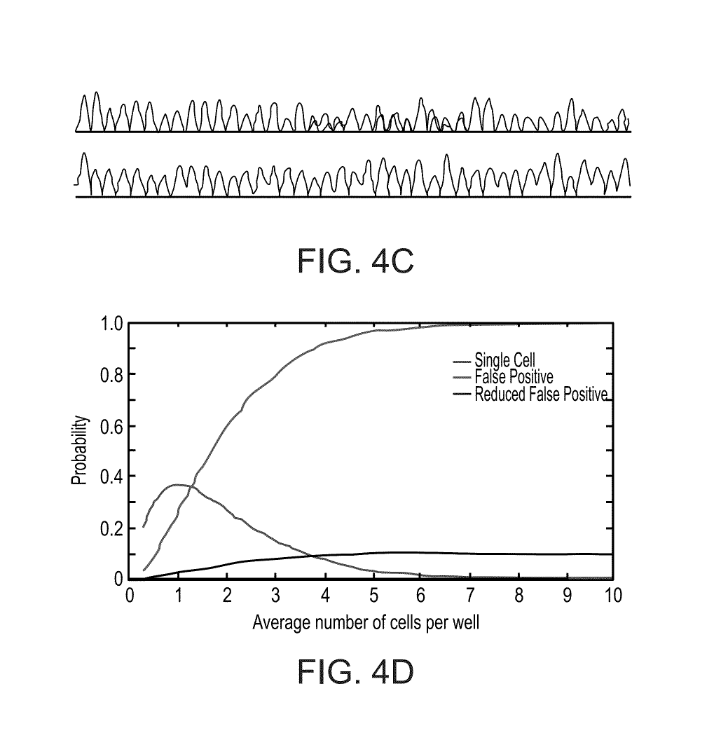

A method which employs fluorescent bacteria that report as to whether the bacteria are of monoclonal or polyclonal origin was developed. The competent cells used were a mixture of different fluorescent bacteria, each expressing a different fluorescent protein. As a result, false positive wells (of polyclonal origin) have, with high probability, a mixed fluorescent signature resulting from the fluorescence of different bacteria. In contrast, true positive wells (of monoclonal origin) have a single fluorescent signature corresponding to the fluorescence of the founder cell of the culture. To this end, several bacterial cultures, each expressing a different fluorescent protein¹⁴, were established in E. coli. The fluorescent signatures measurements of bacteria expressing different fluorescent proteins (e.g Cherry, Citrine etc.) were carried out in 96/384 well plates (See FIGS. 10A-E) and the unique fluorescence emission signature from each culture was recorded for future reference. The fluorescent bacteria were then mixed in equal concentrations, made competent for transformation (See Methods) and used in cloning experiments. Cultures produced with the competent fluorescent bacteria and the cloning method were used to test their usefulness. The fluorescence signature of clones cultures was measured and predicted for each whether they are polyclonal or monoclonal by comparing their fluorescence signature to the reference signature of fluorescent monoclonal cultures. The cultures were then sequenced and the predictions were shown to be correct. As evident by FIG. 4C, there is a certain degree of overlap between the fluorescent spectra of some of these proteins. Nevertheless, this overlap is relatively small and does not compromise the ability to detect whether the signal is from a monoclonal culture, since this protein bleeds into the wavelength of other proteins in a unique and predictable manner. This produces a unique fluorescent signature for this protein which aids in its identification. In these experiments a mixture of four different fluorescent reporter bacteria was used. Nevertheless, the variety of fluorescent reporter bacteria can be further expanded with other existing proteins¹³ if a higher degree of certainty is required. Verification of clonality using fluorescent bacteria enables us to inoculate single cells with higher densities in multi-well plates without having to pay the price of processing false positive (polyclonal) wells (See FIG. 4D).

### Example 4

### Automation

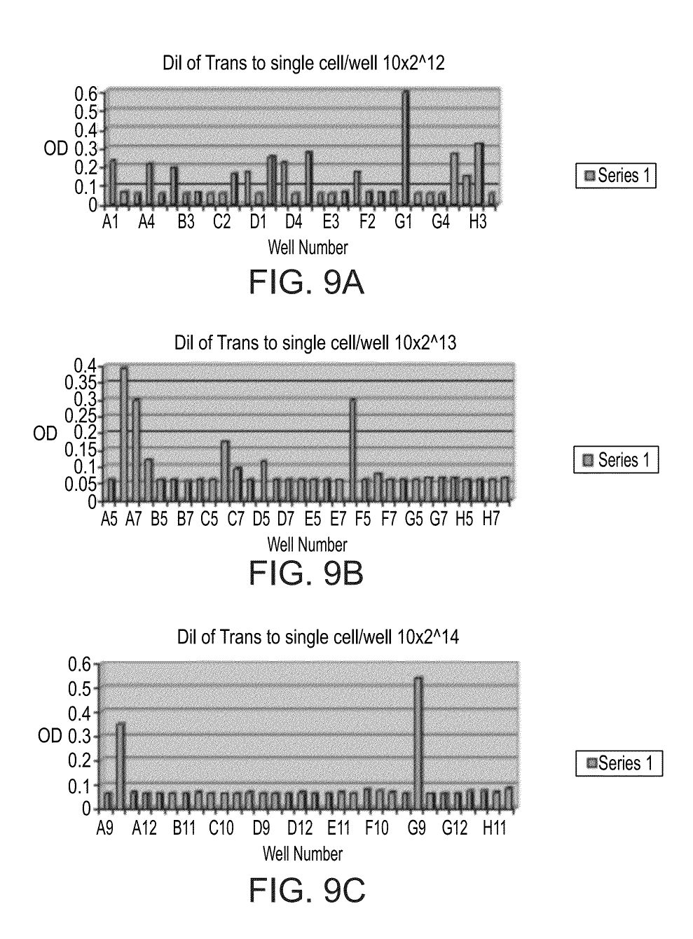

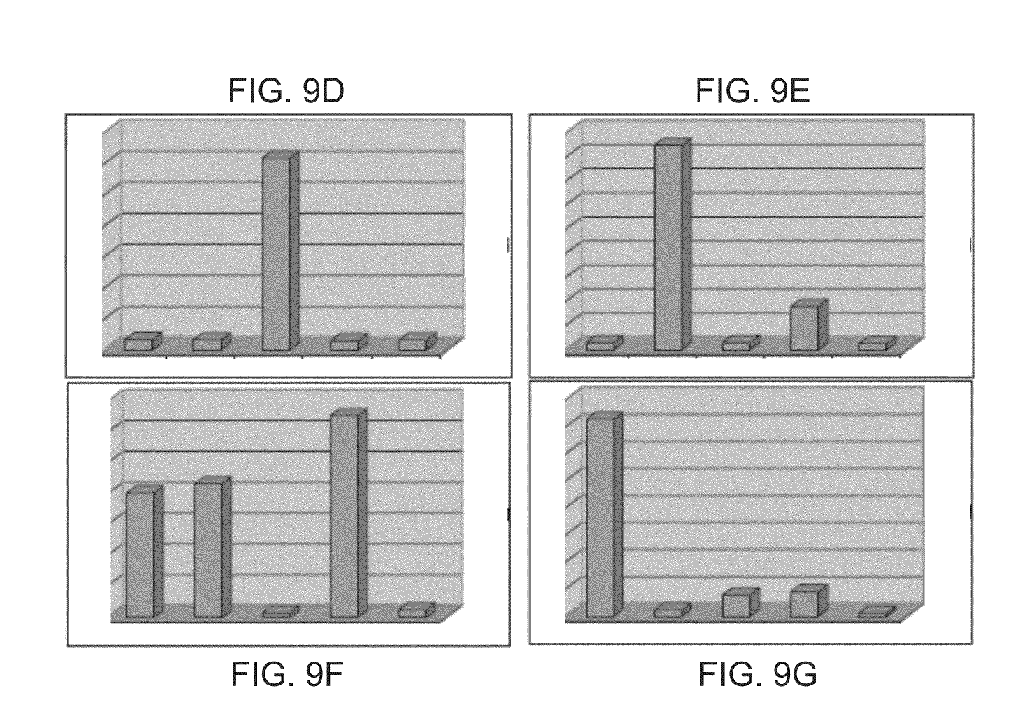

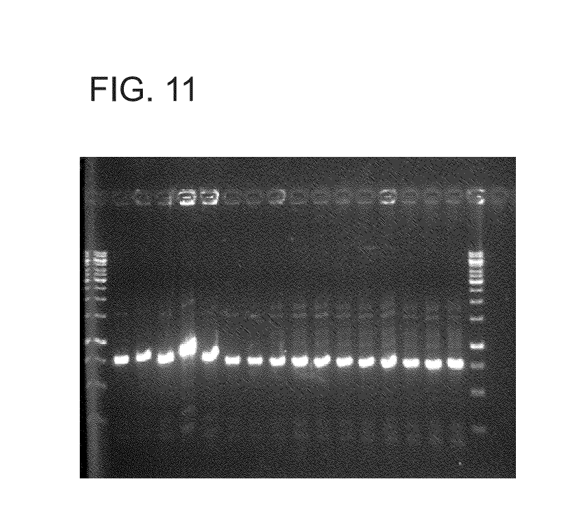

The basics of the present cloning method were designed to be compatible with automation using standard off-the-shelf components. To this end, a Tecan 2000 liquid handling system was programmed using in-house developed robot control software (See FIGS. 7-11) to carry out cloning in an automated manner. Automated procedures were developed for the entire process including ligation, transformation, clonal amplification, plasmid purification and DNA sequencing (See Methods). Automated hardware used included a Liquid Handling arm (LiHa), a Robotic Manipulator Arm (RoMa), automated plate reader, centrifuge, plate shaker and incubator. In addition, control and analysis of results was performed in real time with automatically generated robotic scripts according to real time results. Examples of specific robotic scripts used for various cloning steps can be found below. The plating of clones into 384-well plates is executed at a rate of approximately 8 wells/second, thereby producing two clones per second in multi-well plates (using a ratio of 1/3). At this rate, 96 individually addressable clones are plated in under a minute and 3000 separated and individually addressable clones are plated in under an hour. The system can in principle execute the method at the same rate and accuracy day in day out.

### Example 5

### Computer Aided Cloning and Sequencing of a DNA Library

In order to demonstrate the utility of the present cloning method in high throughput research a very large synthetic DNA library was cloned and sequenced. To this end, 32 automated transformations were performed (See Methods) of a synthetic bar-coded DNA library into competent E. coli cells (Z competent, Zymo research, See Methods). Each transformation is of a diverse population of DNA molecules from which many clones should be made and sequenced. The cloning procedure for this library was executed according to the procedures described before. The clones were dispersed in the inoculation plates with a positive/negative ratio of 1/3. A total of 768 wells (2 separate 384 well plates) were inoculated with this ratio. Automated colony picking was performed as described before and each colony was grown in 2 ml of LB for overnight growth. Plasmid DNA was extracted using an automated procedure (See Methods) from a random sample of 96 of the positive wells and sequenced, also using an automated sequencing procedure (see Methods). In this demonstration it was decided not to clone or sequence with higher throughput (thousands scale) since it is not necessary for illustrative purposes alone. The sequencing results of the bar-coded GFP library show that positive wells were indeed monoclonal colonies (see FIG. 4b) and were of the DNA library that was cloned. Only 2 plasmids out of the 96 sequenced propagated more than one molecule (see FIG. 4b) and the rest propagated one plasmid. These two clones likely reflect the natural rate of double plasmid transformation¹⁵. Throughput in the hundreds to thousands (sequenced clones) scale per batch can also be accomplished by starting with more transformations and/or plating more 384-well plates.

### Example 6

### Colony PCR for Computer Aided Cloning in Liquid

PCR sequencing/screening of clones directly from culture has obvious advantages compared to first having to isolate DNA. Nevertheless the utility of the technique remains limited due to the inherent limitations associated with its manual preparation. The most critical limitations of this method are the variability in the amount of cells and culture media taken into each PCR using manual picking of clones from Petri dishes. Taq DNA polymerase is easily inhibited by debris from bacterial cells and components of culture media, and therefore, inconsistent results are often obtained. Embodiments of the present method confine the cloning procedure to liquid media, enabling standardization of the number of cells and amount of culture media inserted into each PCR. The parameters relevant to performing colony PCR from liquid media were optimized (See Methods). The present analysis shows that when using these optimized conditions, the success rate of colony PCR and sequencing is comparable to that of isolating DNA from clones and sequencing it. This enables robust high-throughput PCR sequencing/screening of cloned libraries without the inherent limitations associated with colony PCR through colony picking.

The quest for high throughput is one of the most influential trends in modern biological research, continuously transforming the way research is planned and conducted. Nevertheless, biology is only at the outset of its high-throughput era. The establishments of new techniques for bacterial cloning more suited to high throughput will facilitate faster movement beyond its gates. Traditional cloning into Petri dishes is difficult to upscale and automate for the average lab and is one of the reasons that the throughput of bacterial cloning has stagnated. Bacterial cloning by dilution, originally demonstrated for the first time over a century ago, was neither intended nor applicable for high throughput. However, it is suggested that the basic principles of cloning by dilution better adapted to the requirements of high throughput research given the technology available today. The average high throughput oriented lab most likely already has all the equipment required to execute the present method in an automated or semi-automated manner. Labs not oriented towards high throughput research can use the present method manually to extend their current cloning throughput, however will have difficulty reaching the scale of thousands of clones without considerable manual labor.

New cloning methods should ease the experimental burden of generating and processing many clones. Additionally, they should inherently support the banking, analysis and documentation of materials and data from high throughput experiments. For example, in contrast to recently developed high-throughput sequencing technology [454, Ilumina, Solexa] in which the (in vitro cloned) sequenced DNA cannot be physically addressed post sequencing, in high throughput cloning preserving the ability to physically address clones post cloning and analysis is important. In addition, in contrast to the comparison between Sanger and next-generation sequencing technology, in cloning there is no reason for per-clone accuracy to trade off with throughput. It is reasoned that current multi-well plate standards (1536 wells/plate practical limit, 384 in this study) provide a reasonable solution for high throughput cloning and its subsequent analysis, banking and documentation needs. One of the major obstacles facing the developments of future cloning platforms with throughput capabilities higher than those demonstrated here (e.g. similar to the throughput of next-generation sequencing) is the ability to individually physically address each clone. Once such platforms become available, then bacterial cloning could potentially advance even beyond this current upper limit.

### Example 7

### Robot Scripts

Exemplary script for explaining the structure and syntax of an exemplary high level robot control language:

### Script Example

TABLE table_PIE1000.gem*
* A preset definition of the way our working deck is organized
DOC
PCR ON PLASMID FOR Fragments A, B, C & D.**
PURIFYING PCR SAMPLES **
MEASURING CONCENTRATION OF PCRS**
PREPARING C.E AND G.E ANALYSIS **
** verbal documentation for the program
ENDDOC
ADDRESS someone@weizmann.ac.il - defining email and SMS number for
notification of errors during preparation
SMS_NUMBER 0538631334
Defining the reagents and their location

| | |
|---|---|
| REAGENT LB_SYBR | T2 5 LCWAUTOBOT 4 |
| REAGENT DDW | T4 1 LCWAUTOBOT 4 |
| REAGENT PCR_dNTP_Mix_x5 | T1 1 LCWAUTOBOT 1 |
| REAGENT TEMP_PCR_A | T1 5 LCWAUTOBOT 1 |
| REAGENT TEMP_PCR_B | T1 6 LCWAUTOBOT 1 |
| REAGENT TEMP_PCR_C | T1 7 LCWAUTOBOT 1 |
| REAGENT TEMP_PCR_D | T1 8 LCWAUTOBOT 1 |
| LOAD GFP_primers_6 | P2 LCWAUTOBOT |

LIST Reaction_200 _ list of reactions to assemble: specifies reagents and volume of
each reagent. Each line is one reaction

| | | | | |
|---|---|---|---|---|
| GFP1F_1p_FAM | 10 GFP_A_R_1p_phos | 10 | TEMP_PCR_A | 5 |

PCR_dNTP_Mix_x5 6.25

| | | | | |
|---|---|---|---|---|
| GFP_B_F_1p_phos 10 | GFP_B_R_1p | 10 | TEMP_PCR_B | 5 |

PCR_dNTP_Mix_x5 6.25

| | | |
|---|---|---|
| GFP_C_F_1p 10 GFP_C_R_1p_phos 10 | TEMP_PCR_C | 5 |

PCR_dNTP_Mix_x5 6.25

| | | | | |
|---|---|---|---|---|
| GFP_D_F_1p_phos 10 | GFP_D_R_1p | 10 | TEMP_PCR_D | 5 |

PCR_dNTP_Mix_x5 6.25
# NTC

| | | | | |
|---|---|---|---|---|
| GFP1F_1p_FAM | 10 GFP_A_R_1p_phos | 10 | TEMP_PCR_D | 5 |

PCR_dNTP_Mix_x5 6.25

| | | | | |
|---|---|---|---|---|
| GFP_B_F_1p_phos 10 | GFP_B_R_1p | 10 | TEMP_PCR_C | 5 |

PCR_dNTP_Mix_x5 6.25

| | | | | |
|---|---|---|---|---|
| GFP_C_F_1p | 10 GFP_C_R_1p_phos | 10 | TEMP_PCR_B | 5 |

PCR_dNTP_Mix_x5 6.25

| | | | |
|---|---|---|---|
| GFP_D_F_1p_phos 10 GFP_D_R_1p | 10 | TEMP_PCR_A | 5 |

PCR_dNTP_Mix_x5 6.25
ENDLIST
LIST CE_200 - list of sample names for export to the CE analysis machine
lambda1_5
ambda1_6
lambda1_7
lambda1_8
ENDLIST
SCRIPT
PROMPT remove tube covers!!!!! (on screen textual notification for user during robot
operation)
% PREPARING PCR REACTIONS - Command for preparing the list that was
specified in the list section

| | | |
|---|---|---|
| PREPARE_LIST Reaction_200 | P3 A1+8 | DEFAULT |

MIX:LCWMXSLOW:10x8,LOG:R200
% TRANFERING PCR PLATE FROM ITS POSITION TO THE PCR BLOCK AND
BACK
MOVE_PLATE P3 PCR *
MOVE_OBJECT COVER HA7 PCR *
RUN_PCR 3 1 1 1 *
MOVE_OBJECT COVER PCR HA7 *
MOVE_PLATE PCR P3
* Commands for using the robots arm to move plates, plate covers and for operating the

PCR block.

% PCR PURIFICATION - command for purifying samples with vacuum based
purficiation scheme
PCR_PURE P3 A1+8 V1 A1+8 P3 A2+8 DDW 31 60 PCR_PURE
% PREPARING MEASURMENT OF DNA BY PICO-GREEN FLUORESCENCE -
Command for measuring DNAconc. Using the picogreen reagent and a table-top
fluorimeter
PG_PREPARE_STD P4 A1+8
PG_PREPARE_SAMPLE P3 A2+8 P4 A2+8 3
% GENERATING A FRAGMENT ANALYSIS C.E RUN - Command for generating a
C.E analysis experiment for the samples specified in the list before (CE_200)
CEPLATE Gr_GFP_React_200 CE_200 A1 Gr_Data
FA_50_POP4_Time3500_Temp60_InjVolt1.0_InjTime20
% PREPARING SAMPLES FOR GEL ELECTROPHORESIS

| | |
|---|---|
| DIST_REAGENT LB_SYBR P6 A1+8 | 5 DEFAULT LOG:SYBR * |

* Command for distributing one reagent to different destinations (loading buffer)
TRANSFER_WELLS P3 A2+8 P6 A1+8 6 LCWBOT **
** Command for transferring a volume of liquid from source wells to destination wells
ENDSCRIPT - End of program

The following includes key robot control scripts used in the automated cloning method.

### Transformation and Dilution

TABLE Table_Clone96
DOC
Add negative control to E1 32 −> Transformation with no DNA
Positions used:
P3 - Optical plate for plate reader
P10 = Microplate for serial dilution
P6 = Strip (of 32) with competent cells
P4 - Deep well for recovery and final dilution
P5 - LB Bucket
P11- Microplate for serial dilution
ENDDOC
#GLOBAL REAGENTS
REAGENT LB_Bucket P5 A5 DEFAULT 8
LABWARE dilutionplate1 P13 “96 Well PCR Plate”
LABWARE dilutionplate2 P14 “96 Well PCR Plate”
%===============================
############ SCRIPT SECTION ###############################
SCRIPT

| | |
|---|---|
| ######## | Transformation |

| | |
|---|---|
| PROMPT | Put 31 plasmids at E1+31 and DDW at E1 32 (Negative control)! |

PROMPT you need plate LB Bucket with AMP at p5, Deep well for dilutions at P4,
Optical plate for plate reader at p3.

| | |
|---|---|
| DIST_REAGENT2 LB_Bucket P4:A1+32 | 950 PIE_AUTAIR |

TIPTYPE:1000

| | |
|---|---|
| PROMPT | Move cells from ice to robot ( 1-16 IN P6) and IMMEDIATELY start |

the script (DNA addition) !
# Thaw competent cells plate on ice (or 0-2C) and place at P6
# Mix DNA with cells (50ul) - not more than 5%
TRANSFER_WELLS E1 1+16 P6 A1+16 3 PIE_BOTBOT
TIPTYPE:50,MIX:PIE_MIX_AUT:4x7
PROMPT TAKE THE CELLS (1-16) BACK TO ICE AND PUT THE OTHER CELLS
(17-32) ON THE ROBOT!!!
TRANSFER_WELLS E1 17+16 P6 A3+16 3 PIE_BOTBOT
TIPTYPE:50,MIX:PIE_MIX_AUT:4x7

| | |
|---|---|
| PROMPT | Gently tap with fingers and IMMEDIATELY MOVE STRIP TO ICE |

and press Enter for a 20 min count !
WAIT 1200

| | |
|---|---|
| PROMPT | MOVE STRIP BACK TO P6 ! |

| | |
|---|---|
| TRANSFER_WELLS P6 A1+32 | P4 A1+32 50 PIE_BOTBOT |

TIPTYPE:50
MIX_WELLS P4 A1+32 10 150 PIE_MIX_AUT TIPTYPE:200
# Dilution of transformed cells 1:43

| | | |
|---|---|---|
| DIST_REAGENT2 LB_Bucket | P3:A1+32 | 293 PIE_AUTAIR |

TIPTYPE:1000,TIPMODE:KEEPTIP

| | |
|---|---|
| TRANSFER_WELLS P4 A1+32 P3 A1+32 | 7 PIE_AUTAIR |

TIPTYPE:200,MIX:PIE_MIX_AUT:2x150
# Negative Control

| | | |
|---|---|---|
| DIST_REAGENT2 LB_Bucket | P3:A5+8 | 300 PIE_AUTAIR |

TIPTYPE:1000
# End dilution of transformed cells 1:43

| | |
|---|---|
| ######## | Growth to fixed OD |

| | |
|---|---|
| PROMPT | Move to plate reader for monitored growth |
| PROMPT | Move plate back from plate reader to robot |

| | |
|---|---|
| ######## | Dill to single cells |

PROMPT you need Microplate for serial dilution at P10, P11,P13 AND P14, old Deep
well at p4, LB_Bucket AMP at p5, old optical plate at p3
# Ditribution of 185 LB into wells for serial dilution
DIST_REAGENT2 LB_Bucket P10:A1+48 149.4 PIE_AUTAIR
TIPTYPE:1000,TIPMODE:KEEPTIP
DIST_REAGENT2 LB_Bucket P10:A7+16 90 PIE_AUTAIR
TIPTYPE:200,TIPMODE:KEEPTIP
DIST_REAGENT2 LB_Bucket P11:A1+48 100 PIE_AUTAIR
TIPTYPE:200,TIPMODE:KEEPTIP
# Dill 1:50,000 Via 4*(10.6 into 149.4)

| | | |
|---|---|---|
| TRANSFER_WELLS P3 A1+8 | P10 A1+8 | 10.6 PIE_BOT AIR |

TIPTYPE:200,MIX:PIE_MIX_AUT:7x120

| | | |
|---|---|---|
| TRANSFER_WELLS P10 A1+8 | P10 A3+8 | 10.6 PIE_BOT AIR |

TIPTYPE:200,MIX:PIE_MIX_AUT:7x120

| | | |
|---|---|---|
| TRANSFER_WELLS P10 A3+8 | P10 A5+8 | 10.6 PIE_BOT AIR |

TIPTYPE:200,MIX:PIE_MIX_AUT:7x120

| | | |
|---|---|---|
| TRANSFER_WELLS P10 A5+8 | P10 A7+8 | 60 PIE_BOTAIR |

TIPTYPE:200,MIX:PIE_MIX_AUT:7x120

| | | |
|---|---|---|
| TRANSFER_WELLS P3 A2+8 | P10 A2+8 | 10.6 PIE_BOT AIR |

TIPTYPE:200,MIX:PIE_MIX_AUT:7x120

| | | |
|---|---|---|
| TRANSFER_WELLS P10 A2+8 | P10 A4+8 | 10.6 PIE_BOT AIR |

TIPTYPE:200,MIX:PIE_MIX_AUT:7x120

| | | |
|---|---|---|
| TRANSFER_WELLS P10 A4+8 | P10 A6+8 | 10.6 PIE_BOT AIR |

TIPTYPE:200,MIX:PIE_MIX_AUT:7x120

| | | |
|---|---|---|
| TRANSFER_WELLS P10 A6+8 | P10 A8+8 | 60 PIE_BOTAIR |

TIPTYPE:200,MIX:PIE_MIX_AUT:7x120
# Dill 1:1,350,000

| | | |
|---|---|---|
| TRANSFER_WELLS P10 A7+8 | P11 A1+8 | 50 PIE_BOTAIR |

TIPTYPE:200,MIX:PIE_MIX_AUT:7x120

| | | |
|---|---|---|
| TRANSFER_WELLS P10 A8+8 | P11 A2+8 | 50 PIE_BOTAIR |

TIPTYPE:200,MIX:PIE_MIX_AUT:7x120

| | | |
|---|---|---|
| TRANSFER_WELLS P11 A1+8 | P11 A3+8 | 50 PIE_BOTAIR |

TIPTYPE:200,MIX:PIE_MIX_AUT:7x120

| | | |
|---|---|---|
| TRANSFER_WELLS P11 A2+8 | P11 A4+8 | 50 PIE_BOTAIR |

TIPTYPE:200,MIX:PIE_MIX_AUT:7x120

| | | |
|---|---|---|
| TRANSFER_WELLS P11 A3+8 | P11 A5+8 | 50 PIE_BOTAIR |

TIPTYPE:200,MIX:PIE_MIX_AUT:7x120

| | | |
|---|---|---|
| TRANSFER_WELLS P11 A4+8 | P11 A6+8 | 50 PIE_BOTAIR |

TIPTYPE:200,MIX:PIE_MIX_AUT:7x120
####### DILUTION OF THE EXEMPLE NUMBER 17-32#################
# Ditribution of 185 LB into wells for serial dilution
DIST_REAGENT2 LB_Bucket P13:A1+48 149.4 PIE_AUTAIR
TIPTYPE:1000,TIPMODE:KEEPTIP
DIST_REAGENT2 LB_Bucket P13:A7+16 90 PIE_AUTAIR
TIPTYPE:200,TIPMODE:KEEPTIP
DIST_REAGENT2 LB_Bucket P14:A1+48 100 PIE_AUTAIR
TIPTYPE:200,TIPMODE:KEEPTIP
# Dill 1:50,000 Via 4*(10.6 into 149.4)

| | | |
|---|---|---|
| TRANSFER_WELLS P3 A3+8 | P13 A1+8 | 10.6 PIE_BOTAIR |

TIPTYPE:200,MIX:PIE_MIX_AUT:7x120

| | | |
|---|---|---|
| TRANSFER_WELLS P13 A1+8 | P13 A3+8 | 10.6 PIE_BOT AIR |

TIPTYPE:200,MIX:PIE_MIX_AUT:7x120

| | | |
|---|---|---|
| TRANSFER_WELLS P13 A3+8 | P13 A5+8 | 10.6 PIE_BOT AIR |

TIPTYPE:200,MIX:PIE_MIX_AUT:7x120

| | | |
|---|---|---|
| TRANSFER_WELLS P13 A5+8 | P13 A7+8 | 60 PIE_BOTAIR |

TIPTYPE:200,MIX:PIE_MIX_AUT:7x120

| | | |
|---|---|---|
| TRANSFER_WELLS P3 A4+8 | P13 A2+8 | 10.6 PIE_BOTAIR |

TIPTYPE:200,MIX:PIE_MIX_AUT:7x120

| | | |
|---|---|---|
| TRANSFER_WELLS P13 A2+8 | P13 A4+8 | 10.6 PIE_BOT AIR |

TIPTYPE:200,MIX:PIE_MIX_AUT:7x120

| | | |
|---|---|---|
| TRANSFER_WELLS P13 A4+8 | P13 A6+8 | 10.6 PIE_BOT AIR |

TIPTYPE:200,MIX:PIE_MIX_AUT:7x120

| | | |
|---|---|---|
| TRANSFER_WELLS P13 A6+8 | P13 A8+8 | 60 PIE_BOTAIR |

TIPTYPE:200,MIX:PIE_MIX_AUT:7x120
# Dill 1:1,350,000

| | | |
|---|---|---|
| TRANSFER_WELLS P13 A7+8 | P14 A1+8 | 50 PIE_BOTAIR |

TIPTYPE:200,MIX:PIE_MIX_AUT:7x120

| | | |
|---|---|---|
| TRANSFER_WELLS P13 A8+8 | P14 A2+8 | 50 PIE_BOTAIR |

TIPTYPE:200,MIX:PIE_MIX_AUT:7x120

| | | |
|---|---|---|
| TRANSFER_WELLS P14 A1+8 | P14 A3+8 | 50 PIE_BOTAIR |

TIPTYPE:200,MIX:PIE_MIX_AUT:7x120

| | | |
|---|---|---|
| TRANSFER_WELLS P14 A2+8 | P14 A4+8 | 50 PIE_BOTAIR |

TIPTYPE:200,MIX:PIE_MIX_AUT:7x120

| | | |
|---|---|---|
| TRANSFER_WELLS P14 A3+8 | P14 A5+8 | 50 PIE_BOTAIR |

TIPTYPE:200,MIX:PIE_MIX_AUT:7x120

| | | |
|---|---|---|
| TRANSFER_WELLS P14 A4+8 | P14 A6+8 | 50 PIE_BOTAIR |

TIPTYPE:200,MIX:PIE_MIX_AUT:7x120
# Dill 1:10 Via 80ul into 720ul (total dil 1:13,500,000)
DIST_REAGENT2 LB_Bucket P4:A6+32 720 PIE_AUTAIR
TIPTYPE:1000,TIPMODE:KEEPTIP

| | | |
|---|---|---|
| TRANSFER_WELLS P11 A5+16 | P4 A6+16 | 80 PIE_BOTAIR |

TIPTYPE:200,MIX:PIE_MIX_AUT:8x150

| | | |
|---|---|---|
| TRANSFER_WELLS P14 A5+16 | P4 A8+16 | 80 PIE_BOTAIR |

TIPTYPE:200,MIX:PIE_MIX_AUT:8x150
# End Dill Of Templates
ENDSCRIPT

### Inoculation

TABLE Table_Clone384S2
DOC
Positions used:
M3- 384 PLATE
M4 - Deep well for recovery and final dilution
M5 - LB Bucket
ENDDOC
#GLOBAL REAGENTS

| | | | | |
|---|---|---|---|---|
| REAGENT | LB_Bucket | P5 | A5 | DEFAULT 8 |
| REAGENT | SAMPLE | P4 | A6 | DEFAULT 8 |

| | | | | |
|---|---|---|---|---|
| REAGENT | SAMPLE2 | P4 | A7 | DEFAULT 8 |

| | | | | |
|---|---|---|---|---|
| REAGENT | SAMPLE3 | P4 | A8 | DEFAULT 8 |

| | | | | |
|---|---|---|---|---|
| REAGENT | SAMPLE4 | P4 | A9 | DEFAULT 8 |
| #REAGENT | SAMPLE6 | P4 | A11 | DEFAULT 8 |

%==============================
# Table Layout (for liquid handling)
LABWARE DW_1 P4 “96 Well DeepWell square”
LABWARE DW_2 P5 “96 Well DeepWell square”
LABWARE 384_PLATE MS1 “384 Well Plate”
LABWARE 384_PLATE MS2 “384 Well Plate”
############ SCRIPT SECTION ###############################
SCRIPT

| | |
|---|---|
| PROMPT | YOU NEED PLATE 384 AT MS2 & MS 1, LB_BUCKET AT P5, OLD |

DEEP WELL WITH THE SAMPLE AT P4.
# Ditribution of 40ul LB into 384 PLATE
DIST_REAGENT2 LB_Bucket MS1:A1++192 40 PIE_AUTAIR
TIPTYPE:200,TIPMODE:MULTIPIP
DIST_REAGENT2 LB_Bucket MS1:B1++192 40 PIE_AUTAIR
TIPTYPE:200,TIPMODE:MULTIPIP
DIST_REAGENT2 LB_Bucket MS2:A1++192 40 PIE_AUTAIR
TIPTYPE:200,TIPMODE:MULTIPIP
DIST_REAGENT2 LB_Bucket MS2:B1++192 40 PIE_AUTAIR
TIPTYPE:200,TIPMODE:MULTIPIP
MIX_WELLS P4 A6+8 4 420 PIE_AUTAUT_DIL TIPTYPE:1000
DIST_REAGENT2 SAMPLE MS1:A1++160 30 PIE_AUTAIR
TIPTYPE:200,TIPMODE:MULTIPIP
DIST_REAGENT2 LB_Bucket P4:A6+8 120 PIE_AUTAIR
TIPTYPE:200,MIX:PIE_MIX_AUT:4x100
DIST_REAGENT2 SAMPLE MS1:A21++32 30 PIE_AUTAIR
TIPTYPE:200,TIPMODE:MULTIPIP
MIX_WELLS P4 A7+8 4 420 PIE_AUTAUT_DIL TIPTYPE:1000
DIST_REAGENT2 SAMPLE2 MS1:B1++160 30 PIE_AUTAIR
TIPTYPE:200,TIPMODE:MULTIPIP
DIST_REAGENT2 LB_Bucket P4:A7+8 120 PIE_AUTAIR
TIPTYPE:200,MIX:PIE_MIX_AUT:4x100
DIST_REAGENT2 SAMPLE2 MS1:B21++32 30 PIE_AUTAIR
TIPTYPE:200,TIPMODE:MULTIPIP
MIX_WELLS P4 A8+8 4 420 PIE_AUTAUT_DIL TIPTYPE:1000
DIST_REAGENT2 SAMPLE3 MS2:A1++160 30 PIE_AUTAIR
TIPTYPE:200,TIPMODE:MULTIPIP
DIST_REAGENT2 LB_Bucket P4:A8+8 120 PIE_AUTAIR
TIPTYPE:200,MIX:PIE_MIX_AUT:4x100
DIST_REAGENT2 SAMPLE3 MS2:A21++32 30 PIE_AUTAIR
TIPTYPE:200,TIPMODE:MULTIPIP
MIX_WELLS P4 A9+8 4 420 PIE_AUTAUT_DIL TIPTYPE:1000
DIST_REAGENT2 SAMPLE4 MS2:B1++160 30 PIE_AUTAIR
TIPTYPE:200,TIPMODE:MULTIPIP
DIST_REAGENT2 LB_Bucket P4:A9+8 120 PIE_AUTAIR
TIPTYPE:200,MIX:PIE_MIX_AUT:4x100
DIST_REAGENT2 SAMPLE4 MS2:B21++32 30 PIE_AUTAIR
TIPTYPE:200,TIPMODE:MULTIPIP
ENDSCRIPT

### Overnight Growth

TABLE Table_Clone384S2
DOC
Positions used:
MS1- 384 PLATE
MS2- 384 PLATE
MS3- 384 PLATE
#M4 - Deep well for recovery and final dilution
P5 - LB Bucket
ENDDOC
#GLOBAL REAGENTS
REAGENT LB_Bucket P5 A5 DEFAULT 8
%===============================
# Table Layout (for liquid handling)
LABWARE LB_BUCKET P5 ″96 Well DeepWell square″
LABWARE 384_1 MS1 ″384 Well Plate″
LABWARE 384_2 MS2 ″384 Well Plate″
LABWARE 16_TUBES1 TS4 ″Tube 13*100mm 16 Pos″
LABWARE 16_TUBES2 TS5 ″Tube 13*100mm 16 Pos″
############ SCRIPT SECTION ###############################
SCRIPT
PROMPT YOU NEED PLATE 384 AT MS2 & MS1, LB_BUCKET AT P5,32
TUBES at TS4 AND TS5.
# Ditribution of 40ul LB into 384 PLATE
DIST_REAGENT2 LB_Bucket TS4:1+8 4000 PIE_AUTAIR TIPTYPE:1000
DIST_REAGENT2 LB_Bucket TS4:9+8 4000 PIE_AUTAIR TIPTYPE:1000
DIST_REAGENT2 LB_Bucket TS5:1+8 4000 PIE_AUTAIR TIPTYPE:1000
DIST_REAGENT2 LB_Bucket TS5:9+8 4000 PIE_AUTAIR TIPTYPE:1000
TRANSFER_WELLS MS1
K1,M1,E2,P2,H4,D5,C6,C7,D8,K8,H9,C10,B11,J11,D12,K12 TS4 1+16 20
PIE_BOTAIR TIPTYPE:50
TRANSFER_WELLS MS1
A13,D13,N13,P13,B14,C14,J15,F16,E17,J17,G18,A19,G19,H19,D20,H20 TS5
1+16 20 PIE_BOTAIR TIPTYPE:50
#################################################################
PROMPT CHANGE THE TUBES TO ANOUTHER 32 TUBES
DIST_REAGENT2 LB_Bucket TS4:1+8 4000 PIE_AUTAIR TIPTYPE:1000
DIST_REAGENT2 LB_Bucket TS4:9+8 4000 PIE_AUTAIR TIPTYPE:1000
DIST_REAGENT2 LB_Bucket TS5:1+8 4000 PIE_AUTAIR TIPTYPE:1000
DIST_REAGENT2 LB_Bucket TS5:9+8 4000 PIE_AUTAIR TIPTYPE:1000
TRANSFER_WELLS MS1 L22,P22,A24 TS4 1+3 20 PIE_BOTBOT
TIPTYPE:50
TRANSFER_WELLS MS2 C1,N1,F2,I3,D4,E4,K4,L4,E5,G5,M5,H6,F8 TS4
4+13 20 PIE_BOTAIR TIPTYPE:50
TRANSFER_WELLS MS2
J8,G9,I9,B10,G10,H10,B11,G11,J12,K12,M14,G15,J15,N15,B16,B17 TS5 1+16
20 PIE_BOTAIR TIPTYPE:50
###################################################################
PROMPT CHANGE THE TUBES TO ANOUTHER 32 TUBES
DIST_REAGENT2 LB_Bucket TS4:1+8 4000 PIE_AUTAIR TIPTYPE:1000
DIST_REAGENT2 LB_Bucket TS4:9+8 4000 PIE_AUTAIR TIPTYPE:1000
DIST_REAGENT2 LB_Bucket TS5:1+8 4000 PIE_AUTAIR TIPTYPE:1000
DIST_REAGENT2 LB_Bucket TS5:9+8 4000 PIE_AUTAIR TIPTYPE:1000
TRANSFER_WELLS MS2 E19,H19,I19,J19,C20,H21,L21,L22,K23,I24 TS4 1+10
20 PIE_BOTAIR TIPTYPE:50
#CHANGE THE MS2 384 PLATE IN THE OLD ONE FROME PREVIOUS
EXPIREMENT.
PROMPT CHANGE THE TWO MS2 PLATES:) ITS THE END:)
TRANSFER_WELLS MS2 F1,B2,L2,M2,N3,F5 TS4 11+6 20 PIE_BOTAIR
TIPTYPE:50
TRANSFER_WELLS MS2
H5,D6,O7,B8,D8,J9,H10,I11,N11,K12,H13,C15,I16,I17,O17,O18 TS5 1+16 20
PIE_BOTAIR TIPTYPE:50
ENDSCRIPT

### Plasmid Purification

```
############ TABLE ###############################
TABLE TABLE_CRUDE
############ HEADER ###############################
P4- 96 DEEP WELL WITH BACTERIA (2.2 ML)
P11 - 96 WELL FILTER PLATE ON TOP OF PCR PLATE.
*P11 - 96 WELL BINDING PLATE ON TOP OF PCR PLATE. (For Zymo..)
T10- 2 EPPENDORF WITH 1 ML EB (3,4)
P5 (on Pilly)- 96 WELL COLLECTION PLATE
P4 (on Pilly) - (96 DEEPWELL FOR FILTERATION)
BUF12- RESUSPENSION BUFFER AT A1, LYSIS BUFFER AT A2,
NETRULIZATION BUFFER AT A3, WASH BUFFER AT A4.
REAGENT Wash_Buff BUF12 25 PIE_AUTBOT_SLOW 8
REAGENT EB_Buff T10 1 PIE_AUTBOT 4
REAGENT DDW P2 1 PIE_TROUGH_AUTAIR 8
CLEANPCR_SAMPLE_VOL = 650
CLEANPCR_WASH_VOL = 500
CLEANPCR_ELUTION_VOL = 50
# Defenitions of Labwares
LABWARE InputSamples P4 “96 Well DeepWell square”
LABWARE FilteredSamples P5 “96 Well DeepWell square”
LABWARE FILTER P11 “96 Well Zymo On PCR Plate”
LABWARE Eppendorf T10 “Block Eppendorf 24 Pos”
#LABWARE Water P2 “6 pos DeepWell trough”
  ############ SCRIPT SECTION ###############################
SCRIPT
# BUF12 - A1: solution_1, A2: solution_2, A3: solution_2, A4:WASH BUFFER
LINKER_POS A
PROMPT Make sure Deep Well 1(WITH SAMPLE) is located at P4
PROMPT Make sure Inputs Trouph (Buffers) is located at BUF12,Waste Trouph
(empty) at PS.Make sure colonge in P11.
PROMPT Make sure that 3 weight (P8: 160g,P7: 180g,P6: 250g) and collection (DW)
(P4) are in position at 2nd Robot and the rest of the table is free.
PROMPT Make sure APIServer is Runing on 2nd Robot and Evoware on 2nd Robot is
not running any script
PROMPT Make Sure linker is free
  # Fill empty wells in plate with Water
#DIST_REAGENT2 DDW P4:A7+48 900 PIE_TROUGH_AUTAIR
TIPTYPE:1000,TIPMODE:KEEPTIP
  # Take Plate to centrifuge
TRANSFER_OBJECT P4 LNK
LINKER_POS B
START_TIMER 1
REMOTE LoadPlate
REMOTE WeightScript_2500g_MiniPrep
REMOTE RetrievePlate
WAIT_TIMER 1 5000
# Take Plate back to its place
LINKER_POS A
TRANSFER_OBJECT LNK P4
PROMPT EMPTY THE PLATE IN P4
# Suck All from top to waste
#TRANSFER_WELLS P4 A1+48 P3 A1+48 900 PIE_BOTAIR_AspLifted
TIPMODE:KEEPTIP,TIPTYPE:1000
#TRANSFER_WELLS P4 A1+48 P3 A2+48 400 PIE_BOTAIR_AspLifted
TIPMODE:KEEPTIP,TIPTYPE:1000
#Transfer solution #1 to Deep Well #1 and mix
GET_TIPS 8 1000
TRANSFER_WELLS BUF12 A1+8x6 P4 A1+48 250 PIE_TROUGH_AUTAIR
TIPTYPE:1000,TIPMODE:NOTIP
DROP_TIPS
GET_TIPS 8 1000
TRANSFER_WELLS BUF12 A1+8x6 P4 A7+48 250 PIE_TROUGH_AUTAIR
TIPTYPE:1000,TIPMODE:NOTIP
DROP_TIPS
##########FOR PCR COLONY#########
GET_TIPS 8 1000
TRANSFER_WELLS BUF12 A1+8 P5 A1+8 250 PIE_TROUGH_AUTAIR
TIPTYPE:1000,TIPMODE:NOTIP
DROP_TIPS
###################################
#### without mix insted we take them to eran sagl to do vortex. (MIX:CRDMX:40x200
)
#need to do vortex insted of the mix.
#GET_TIPS 8 1000
#TRANSFER_WELLS BUF12 A1+8 P4 A2+8 250 PIE_TROUGH_AUTAIR
TIPTYPE:1000,TIPMODE:NOTIP
###MIX:CRDMX:40x200
#TRANSFER_WELLS P4 A2+8 P4 A3+8 250 PIE_BOTAIR
TIPTYPE:1000,TIPMODE:NOTIP
#DROP_TIPS
#GET_TIPS 8 1000
#TRANSFER_WELLS BUF12 A1+8 P4 A4+8 250 PIE_TROUGH_AUTAIR
TIPTYPE:1000,TIPMODE:NOTIP
#TRANSFER_WELLS P4 A4+8 P4 A5+8 250 PIE_BOTAIR
TIPTYPE:1000,TIPMODE:NOTIP
#TRANSFER_WELLS P4 A5+8 P4 A6+8 250 PIE_BOTAIR
TIPTYPE:1000,TIPMODE:NOTIP
#DROP_TIPS
PROMPT Check Suspension
#Transfer solution #2 to Deep Well.
#THIS STAGE SHOULD TAKE 5 MIN.
TRANSFER_WELLS BUF12 A2+8 P4 A1+8 250 PIE_AUTAIR_DISP_7mm_DOWN
TIPTYPE:1000
TRANSFER_WELLS BUF12 A2+8 P4 A2+8 250 PIE_AUTAIR_DISP_7mm_DOWN
TIPTYPE:1000
TRANSFER_WELLS BUF12 A2+8 P4 A3+8 250 PIE_AUTAIR_DISP_7mm_DOWN
TIPTYPE:1000
TRANSFER_WELLS BUF12 A2+8 P4 A4+8 250 PIE_AUTAIR_DISP_7mm_DOWN
TIPTYPE:1000
TRANSFER_WELLS BUF12 A2+8 P4 A5+8 250 PIE_AUTAIR_DISP_7mm_DOWN
TIPTYPE:1000
TRANSFER_WELLS BUF12 A2+8 P4 A6+8 250 PIE_AUTAIR_DISP_7mm_DOWN
TIPTYPE:1000
TRANSFER_WELLS BUF12 A2+8 P4 A7+8 250 PIE_AUTAIR_DISP_7mm_DOWN
TIPTYPE:1000
TRANSFER_WELLS BUF12 A2+8 P4 A8+8 250 PIE_AUTAIR_DISP_7mm_DOWN
TIPTYPE:1000
TRANSFER_WELLS BUF12 A2+8 P4 A9+8 250 PIE_AUTAIR_DISP_7mm_DOWN
TIPTYPE:1000
TRANSFER_WELLS BUF12 A2+8 P4 A10+8 250
PIE_AUTAIR_DISP_7mm_DOWN TIPTYPE:1000
TRANSFER_WELLS BUF12 A2+8 P4 A11+8 250
PIE_AUTAIR_DISP_7mm_DOWN TIPTYPE:1000
TRANSFER_WELLS BUF12 A2+8 P4 A12+8 250
PIE_AUTAIR_DISP_7mm_DOWN TIPTYPE:1000
PROMPT TAKE THE PLATE COVER IT WITH A PAD AND MIX IT BY
INVERTING THE PLATE UP SIDE DOWN 4-6 TIMES.
#Transfer solution #3 and shake
TRANSFER_WELLS BUF12 A3+8 P4 A1+8 250 PIE_AUTAIR_DISP_7mm_DOWN
TIPTYPE:1000
TRANSFER_WELLS BUF12 A3+8 P4 A2+8 250 PIE_AUTAIR_DISP_7mm_DOWN
TIPTYPE:1000
TRANSFER_WELLS BUF12 A3+8 P4 A3+8 250 PIE_AUTAIR_DISP_7mm_DOWN
TIPTYPE:1000
TRANSFER_WELLS BUF12 A3+8 P4 A4+8 250 PIE_AUTAIR_DISP_7mm_DOWN
TIPTYPE:1000
TRANSFER_WELLS BUF12 A3+8 P4 A5+8 250 PIE_AUTAIR_DISP_7mm_DOWN
TIPTYPE:1000
TRANSFER_WELLS BUF12 A3+8 P4 A6+8 250 PIE_AUTAIR_DISP_7mm_DOWN
TIPTYPE:1000
TRANSFER_WELLS BUF12 A3+8 P4 A7+8 250 PIE_AUTAIR_DISP_7mm_DOWN
TIPTYPE:1000
TRANSFER_WELLS BUF12 A3+8 P4 A8+8 250 PIE_AUTAIR_DISP_7mm_DOWN
TIPTYPE:1000
TRANSFER_WELLS BUF12 A3+8 P4 A9+8 250 PIE_AUTAIR_DISP_7mm_DOWN
TIPTYPE:1000
TRANSFER_WELLS BUF12 A3+8 P4 A10+8 250
PIE_AUTAIR_DISP_7mm_DOWN TIPTYPE:1000
TRANSFER_WELLS BUF12 A3+8 P4 A11+8 250
PIE_AUTAIR_DISP_7mm_DOWN TIPTYPE:1000
TRANSFER_WELLS BUF12 A3+8 P4 A12+8 250
PIE_AUTAIR_DISP_7mm_DOWN TIPTYPE:1000
PROMPT TAKE THE PLATE, COVER IT WITH A PAD AND MIX IT BY
INVERTING THE PLATE UP SIDE DOWN 6-8 TIMES.
PROMPT LEAVE THE PLATE IN ROOM TEMP FOR 5 MIN.
WAIT 300
####CENTRIFUGE 2500Xg FOR 5 MIN
## Take Deep Well #3 to centrifuge
TRANSFER_OBJECT P4 LNK
LINKER_POS B
START_TIMER 1
REMOTE LoadPlate
REMOTE WeightScript_2500g_MiniPrep_2nd
REMOTE RetrievePlate
WAIT_TIMER 1 1000
## Take Back DeepWell #3
LINKER_POS A
TRANSFER_OBJECT LNK P4
############################################################
FILTERING THE LYSATE AND BINDING PLASMID
#####################################################
#TRANSFER THE SUPERNATANT TO THE FILTER PLATE (650 UL INSTED OF
THE 750)
TRANSFER_WELLS P4 A1+8 P11 A1+8 650 PIE_BOTBOT_AspLifted
TIPTYPE:1000
TRANSFER_WELLS P4 A2+8 P11 A2+8 650 PIE_BOTBOT_AspLifted
TIPTYPE:1000
TRANSFER_WELLS P4 A3+8 P11 A3+8 650 PIE_BOTBOT_AspLifted
TIPTYPE:1000
TRANSFER_WELLS P4 A4+8 P11 A4+8 650 PIE_BOTBOT_AspLifted
TIPTYPE:1000
TRANSFER_WELLS P4 A5+8 P11 A5+8 650 PIE_BOTBOT_AspLifted
TIPTYPE:1000
TRANSFER_WELLS P4 A6+8 P11 A6+8 650 PIE_BOTBOT_AspLifted
TIPTYPE:1000
TRANSFER_WELLS P4 A7+8 P11 A7+8 650 PIE_BOTBOT_AspLifted
TIPTYPE:1000
TRANSFER_WELLS P4 A8+8 P11 A8+8 650 PIE_BOTBOT_AspLifted
TIPTYPE:1000
TRANSFER_WELLS P4 A9+8 P11 A9+8 650 PIE_BOTBOT_AspLifted
TIPTYPE:1000
TRANSFER_WELLS P4 A10+8 P11 A10+8 650 PIE_BOTBOT_AspLifted
TIPTYPE:1000
TRANSFER_WELLS P4 A11+8 P11 A11+8 650 PIE_BOTBOT_AspLifted
TIPTYPE:1000
TRANSFER_WELLS P4 A12+8 P11 A12+8 650 PIE_BOTBOT_AspLifted
TIPTYPE:1000
####CENTIFUGE 3000Xg FOR 10 MIN
#TRANSFER_OBJECT P11 LNK
MOVE_OBJECT PCR P11 LNK
LINKER_POS B
START_TIMER 1
REMOTE LoadFilterOnDW
REMOTE WeightScript_3000g_MiniPrep_3rd
REMOTE RetrieveFilter
WAIT_TIMER 1 1000
LINKER_POS A
MOVE_OBJECT PCR LNK P11
PROMPT Take Plate from P4 On Pilly and return to Sealy in position P5.
PROMPT Put Waste PLate in P4 on Pilly
PROMPT Put New Waste Plate in Pilly in Position P4.
##################################################################
CLEANING #########################################################
PROMPT ##### ZYMO #####
PROMPT Put Binding plate in P11 instead of filteration plate.
AUT_CLEAN_MiniPrep P5:A1+96 P11:A1+96 CLEANPCR_SAMPLE_VOL
CLEANPCR_WASH_VOL CLEANPCR_ELUTION_VOL P11
#### KEEP THE WELL COLLECTION PLATE AT 4 DEGREE FOR 1-2 DAYES
AND IF YOU NEED TO STOREGE IT FOR LONGER PERIUDE PUT IT IN −20
DEGREE.
ENDSCRIPT
```

### Automated Sequencing

TABLE TABLE_SEQ
############ HEADER ###############################
REAGENT SEQ_T7 T10 23 PIE_AUTAIR_PCR 1
##################
REAGENT SEQ_SP6 T10 24 PIE_AUTAIR_PCR 1
##################
REAGENT DDW T10 17 PIE_AUTAIR_PCR 1
############## ddw list
LIST DDWLIST
17.78571429
0
0
0
10.57142857
0
0
0
0
0
0
0
0
0
10.5
0
0
0
0
0
45.64285714
22.42857143
42.57142857
26.21428571
0
0
0
0
0
0
0
13.07142857
15.21428571
0
0
0
0
0
0
0
7.928571429
0
0
0
0
0
24.57142857
18.28571429
0
0
0
0
10.71428571
0
10.14285714
0
39.21428571
21.21428571
0
11.07142857
0
0
0
0
0
0
0
12.64285714
0
0
0
0
35.78571429
0
0
0
0
0
0
0
28.28571429
34.07142857
0
0
0
0
0
0
0
0
0
0
7.857142857
0
0
16.5
ENDLIST
LABWARE pcrplate P6 “96 Well PCR Plate”
LABWARE CollectionPlate P5 “96 Well PCR Plate”
LABWARE pcrplate P11 “96 Well PCR Plate”
LABWARE ddw P12 “1 pos trough”
LABWARE epp T10 “Block Eppendorf 24 Pos”
SCRIPT
###############################################################
# dilution in PCR Plate P11
TRANSFER_LOCATIONS P5:1+96 P6:1+96 25 PIE_BOTBOT TIPTYPE:50
DIST_REAGENT2 DDW P6:1+96 DDWLIST PIE_AUTBOT
TIPTYPE:200,MIX:PIE_MIX_AUT:2x25
#Transfer of Sequencing + primers mix
DIST_REAGENT2 SEQ_T7 P11:1+96 4 PIE_AUTBOT
TIPTYPE:200,TIPMODE:MULTIPIP
#Transfer of diluted plasmids
TRANSFER_LOCATIONS P6:1+96 P11:1+96 11 PIE_BOTBOT
TIPTYPE:20,MIX:PIE_MIX_AUT:2x10
ENDSCRIPT

### Sequencing Purification

TABLE TABLE_SEQ
############ HEADER ###############################
# Sequencing cleaning parameters
#(for 15ul product diluted with 60ul DDW)
# how much to dilute the sample after the reaction, before loading into EdgeBio plate
#DILUTE_BEFORE_VOL = 75
# after should be diluted by 80ul for 16cap and by 40ul for 96cap
#DILUTE_AFTER_VOL = 40
# how much to take from diluted plate into EdgeBio plate
CLEANSEQ_SAMPLE_VOL = 25
CLEANSEQ_WASH_VOL = 150
CLEANSEQ_REFILL_VOL = 300
REAGENT DDW BUF12 9 PIE_TROUGH_AUTAIR 8
######################locations of
plates##############################################
# plates in main robot (Seali):
# P5: Tetrad PCR plate (with samples after sequencing reaction)
# P11: EdgeBio on Tetrad PCR plate
#BUF12: Water trough
# P7: (Only after elution of samples) CE plate
#---------------------------------------------------------------------------------
# plates in second robot (Pilli):
# -------------------------------
# CE plate in location P1 (on stand- SHNEKEL)
weight 200g in location P8
# 1 waste plates (Deep Well with “Edge-Bio” written on them) are in position P4
# capillary plate (on stand) is in position P1
# NO plate on Linker
######################################################################
#################
#LABWARE Tamir P4 “96 Well PCR Plate”
LABWARE Shiran P5 “96 Well PCR Plate”
#LABWARE Shiran2 P6 “96 Well PCR Plate”
#LABWARE Tuval P13 “96 Well PCR Plate”
SCRIPT
###############################################################
# AUT_CLEAN_SEQ parameters:

| | |
|---|---|
| # | 1: Locations of samples |
| # | 2: Wells in Edgebio to use |

| | |
|---|---|
| # | NOTE: Lists must be of equal length |

# Example: To clean sequencing from P6:A1+47 in Edgebio in wells A3+47:
#AUT_CLEAN_SEQ P4:1+20;P5:A7+10,D8+10;P6:A1+28;P13:A1+28 A1+96
AUT_CLEAN_SEQ P5:1+96 A1+96
# Plates used in this script
%% Plate Sample used in pos P6
%% Plate EdgeBio used in pos P11
%% Plate CE in P1 on second robot (Pilli)
ENDSCRIPT

### Example 8

### Assessing Clonality According to the Present Teachings (FIGS. 7-11)

Specifically in high throughput research, estimating CFU after transformation is crucial. This is due to the fact that often large, diverse DNA libraries are transformed and estimating the number of transformed cells testifies to the degree at which the DNA propagated within the transformed bacteria represents the initial DNA library. Additionally, the CFU count is an important internal control to the transformation procedure itself

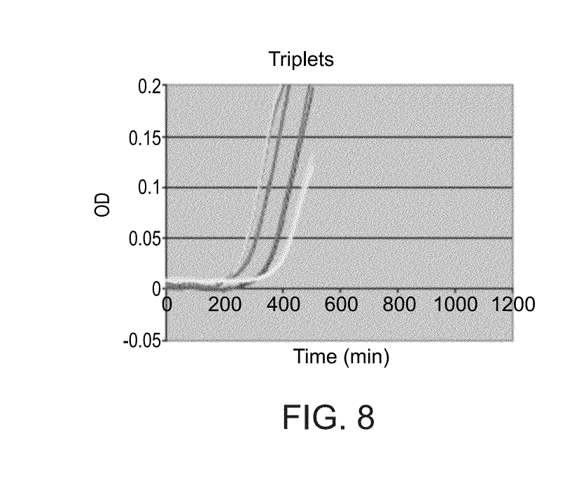

Several batches of transformed bacteria spanning a wide range of CFU values were cultured in multi-well plates inside a heated plate reader with constant OD monitoring. Identical samples from each transformation were simultaneously plated onto Petri dishes and using our new procedure and the CFU values for both was determined. The CFU value resulting from both methods accurately and reproducibly correlates with the time it took the culture to gain a predetermined OD value. Additionally, the plating a 100 fold difference in transformation efficiency. In the comparison between traditional and digital cloning both were performed with the same transformation, ruling out transformation method as a possible factor in increasing the CFU of digital compared to traditional cloning. Therefore, it is the number of transformed cells that survive the process of plating that distinguishes cloning in liquid medium as opposed to plating onto solid medium. A side-by-side comparison between the two techniques was done by simultaneously plating an identical aliquot of transformed bacterial cells immediately following transformation onto both solid medium in Petri dishes and digitally into liquid growth medium in multi-well plates and performed a comprehensive CFU count for both. The CFU value for cells plated and cultured on solid medium in Petri dishes was obtained by regular manual counting of colonies. The CFU value an identical sample of cells in the case of digital cloning was obtained by diluting the transformed cells to well <single viable cell/well concentrations and plating them into 96 well plates. A colony count was then obtained by both a rapid plate reader scan and was verified by a manual count. By diluting the transformed cells to concentrations considerably lower than one viable cell/well it was ensured that each positive well is a clone with high probability. This enabled to perform an accurate digital CFU count on the basis of positive and negative wells in 96 well plates (See FIG. 8). Nevertheless, the clonality of positive wells from the digital CFU count was also verified using DNA sequencing.

Below Table 3 shows the data of the correlation between time to OD and a manual colony count of transformations into Petri dishes. This shows that time-to-OD is linearly correlated to the actual CFU value.

TABLE 3

| Time to | |
|---|---|
| OD (min) | CFU\100 ul |
| 280 | 6656 |
| 311 | 3328 |
| 382 | 1664 |
| 429 | 832 |
| 496 | 416 |
| 575 | 208 |
| 684 | 104 |
| 796 | 52 |
| 894 | 26 |
| 970 | 13 |

TABLE 4
Cloning into LB

| 10 × 2 > 12 | 10 × 2 > 13 | 10 × 2 > 14 | # Clones |
|---|---|---|---|
| Dil1 | Dil2 | Dil3 | 1 |
| A1 | A6 | A10 | 2 |
| A4 | C6 | G9 | 3 |
| B2 | E8 | | 4 |
| C3 | A7 | | 5 |
| C4 | F6 | | 6 |
| D2 | A8 | | 7 |
| D3 | C7 | | 8 |
| E1 | D5 | | 9 |
| F1 | | | 10 |
| G1 | | | 11 |
| H1 | | | 12 |
| H3 | | | 13 |
| H2 | | | |

Transformation efficiency in LB

| | |
|---|---|
| 100 ul were inoculated out of 1 ml of | |
| transformation | |
| The calculation is done according to the 10 * 2 > 12 | |
| dill | |
| Number of colonies in the 10 * 2 > 12 Dil | 13 |
| Number of colonies for in the entire | 5324800 |
| transformation (225 ng) | |
| Number of colonies per ng DNA (CFU/ng) | 23665.77778 |

Cloning into plates

| Number of colonies on plates after 50 ul | Dillution from |
|---|---|
| inoculation | transformation |
| 5 | 10 × 2\hat{ }6 |
| 9 | 10 × 2\hat{ }5 |
| 23 | 10 × 2\hat{ }4 |
| 31 | 10 × 2\hat{ }3 |
| 62 | 10 × 2\hat{ }2 |
| 0 | Neg Con (No DNA) |
| Transformation efficiency in plates | |
| Number of colonies in 50 ul diluted 10 × 2\hat{ }2 | 62 |
| Number of colonies in entire 1 ml transformation | 49600 |
| Number of colonies per ng DNA | 220.4444444 |
| Transformation efficiency in LB | 23665.77778 |
| Transformation efficiency in Plate | 220.4444444 |
| Ratio of LB/Plate efficiencies | 107.3548387 |
| Number of colonies in entire 1 ml transformation | 49600 |
| Number of colonies per ng DNA | 220.4444444 |

Clonality by fluorescence signature and validation by PCR is shown in FIGS. 10A-B to 11.

### Example 8

### Determining the CFU Value of Liquids

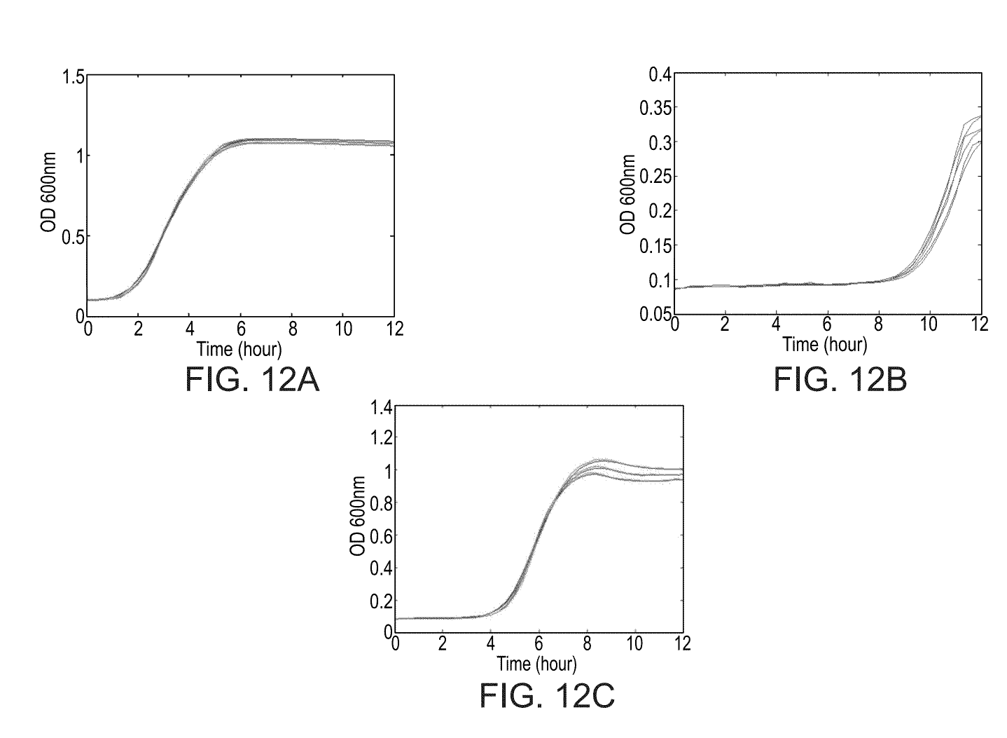

Determining the CFU value of a given liquid (for example urine) is traditionally done by counting the number of colonies that appear after 24-36 hour growth from single cells on a solid growth medium. The present inventors propose to determine CFU and consequently whether a urine sample is contaminated (for UTI diagnosis) without growing cells from single cells and without colony counting. Instead, they propose growing a liquid culture directly from the urine and analyze 2 basic parameters of the monitored growth curve in real-time. The first is (1) the time at which a Urine culture passes a threshold signal to noise ratio (i.e. the time taken to reach the log phase of growth) and the other (2) is the kinetics of the growth curve (i.e. rate of increase of growth during the log phase of growth). Computational learning algorithms, such as naïve based classification are used in order to learn the behavior of these parameters on a training set of a large number of samples and then diagnose new samples according to the knowledge gained from the training set. Monitoring bacterial growth in liquid medium under real-time optical density (OD) monitoring, has given the following results in FIGS. 12A-C and Table 5 herein below.

TABLE 5

| | Turbidity rise | Turbidity rise | Turbidity rise |
|---|---|---|---|
| | <3 hours | >5 hours | 3-5 hours |
| 6-Jul | 1 | 5 | 1 |
| 11-Jul | 1 | 8 | 4 |
| 17-Jul | 1 | 10 | |
| 19-Jul | | 7 | |
| 20-Jul | | 6 | |
| Total | 3 | 36 | 5 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Text

1. Weiss R A 2005. Robert Koch: the grandfather of cloning? Cell; 18; 123(4):539-42.
2. Stanley N. Cohen, Annie C. Y. Chang, and Leslie Hsu 1972. Nonchromosomal Antibiotic Resistance in Bacteria: Genetic Transformation of Escherichia coli by R-Factor DNA Proc Natl Acad Sci; 69(8): 2110-2114.
3. Masanari Kitagawa, Takeshi Ara, Mohammad Arifuzzaman, Tomoko loka-Nakamichi, Eiji Inamoto, Hiromi Toyonaga, and Hirotada Mori 2005. Complete set of ORF clones of Escherichia coli ASKA library: Unique Resources for Biological Research DNA Research; 12, 291-299.
4. Tomoya Baba, Takeshi Ara, Miki Hasegawa, Yuki Takai, Yoshiko Okumura, Miki Baba, Kirill A Datsenko, Masaru Tomita, Barry L Wanner and Hirotada Mori 2006. Construction of Escherichia coli K-12 in-frame, single-gene knockout mutants: the Keio collection Mol Syst Biol; 2:0008.
5. Zaslayer A, Bren A, Ronen M, Itzkovitz S, Kikoin I, Shavit S, Liebermeister W, Surette M G, Alon U. 2006. A comprehensive library of fluorescent transcriptional reporters for Escherichia coli. Nat Methods; 3(8):623-8.
6. Lynda Dieckman, Minyi Gu, Lucy Stols, Mark I. Donnelly, and Frank R. Collart 2002. High Throughput Methods for Gene Cloning and Expression Protein Expression and Purification 25, 1-7.
7. Cox J C, Lape J, Sayed M A, Helling a HW. 2007. Protein fabrication automation. Protein Sci. March; 16(3):379-90.
8. Uber D C, Jaklevic J M, Theil E H, Lishanskaya A, McNeely M R. 1991. Application of robotics and image processing to automated colony picking and arraying. Biotechniques; November; 11(5):642-7.
9. Ben Yehezkel T, Linshiz G, Buaron H, Kaplan S, Shabi U, Shapiro E. 2008. De novo DNA synthesis using single molecule PCR. Nucleic Acids Res; October; 36(17):e107.
10. Jean-Michel Betton, 2004. High throughput cloning and expression strategies for protein production Biochimie; 86 601-605.
11. Breed, R and WD Dotterrer. 1916. The Number of Colonies Allowable On Satisfactory Agar Plates. J. Bacteriol. 1:321-331.
12. Tomasiewicz, D. M. et al. 1980. The Most Suitable Number of Colonies On Plates for Counting. J Food Prot. 43(4):282-286.
13. Linshiz G, Yehezkel T B, Kaplan S, Gronau I, Ravid S, Adar R, Shapiro E. 2008. Recursive construction of perfect DNA molecules from imperfect oligonucleotides. Mol Syst Bio1; 4:191.
14. Shaner N C, Steinbach P A, Tsien R Y. 2005. A guide to choosing fluorescent proteins. Nat Methods; December; 2(12):905-9.
15. A. Weston, G. O. Humphreys, Moira G. M. Brown, and J. R. Saunders 1979. Simultaneous Transformation of Escherichia coli by Pairs of Compatible and Incompatible Plasmid DNA Molecules Molec. gen Genet; 172, 113-118

1. A method of diagnosing an infection in a subject, the method comprising:
(a) culturing a fluid sample of the subject under conditions that allow a pathogen in said sample to propagate in a liquid culture, wherein a propagation of said pathogen comprises an initial lag phase and a subsequent growth phase;
(b) analyzing a rate of increase of an optical density of said liquid culture during said growth phase; and
(c) determining the time required to reach said growth phase;
wherein a combination of said rate of increase above a predetermined level and said time required to reach said growth phase below a predetermined level is indicative of the infection in the subject.

2. The method of claim 1, wherein the infection is a bacterial infection or a fungal infection.

3. (canceled)

4. The method of claim 1, wherein said fluid sample is selected from the group consisting of urine, cerebrospinal fluid, semen, plasma and blood.

5. The method of claim 1, further comprising confirming a result of the diagnosis.

6. The method of claim 1, further comprising informing the subject of a result of the diagnosis.

7. The method of claim 1, wherein said liquid culture comprises culture medium.

8. A method of determining colony forming units (CFU) of a pathogen, the method comprising:
(a) culturing a fluid sample under conditions that allow a pathogen in said sample to propagate in a liquid culture, wherein a propagation of said pathogen comprises an initial lag phase and a subsequent growth phase;
(b) analyzing a rate of increase of an optical density of said liquid culture during said growth phase; and
(c) determining the time required to reach said growth phase;
wherein a combination of said rate of increase and said time required to reach said growth phase correlates to the CFU.

9. A method of determining clonality of a cell culture comprising:
providing a cell culture which comprises cells expressing a plurality of distinct reporter polypeptides, each of said plurality of distinct reporter polypeptides being expressed by different cells of said cell culture,
wherein said plurality of distinct reporter polypeptides are selected:
(i) having distinctive signals;
(ii) generating a distinctive signal when co-expressed in a culture, said distinctive signal being distinguishable from said distinctive signals; and
determining clonality of said cell culture based on expression of said plurality of distinct reporter polypeptides, wherein a presence of said distinctive signal is indicative of a non-clonal culture and wherein an absence of distinctive signal is indicative of a clonal culture.

10. An isolated population of cells comprising competent cells said competent cells expressing an exogenous recombinant polynucleotide encoding a reporter polypeptide.

11. The isolated population of cells of claim 10, wherein said recombinant polynucleotide is not a translational fusion.

12. (canceled)

13. The method of claim 9, wherein said cell culture is a prokaryotic culture.

14. (canceled)

15. The method of claim 9, wherein said cell culture is a eukaryotic culture.

16. The method of claim 9 further comprising diluting said cell culture prior to determining clonality.

17. The method of claim 9, further comprising determining a level of said distinctive signal in a reference culture.

18. The method of claim 9, wherein said reference culture is a polyclonal culture.

19. The method of claim 9, wherein said reference culture is a monoclonal culture.

20. The method of claim 9, wherein at least one of said plurality of distinct reporter polypeptides is not a translational fusion.

21. The method of claim 9, wherein said culture comprises competent cells.

22. The method of claim 9, wherein said cell culture is a liquid culture.

23. The method of claim 9, wherein said cell culture comprises cells transformed with a polynucleotide of interest.

24. (canceled)

25. A method of determining colony forming units (CFU) of a pathogen, the method comprising:
serially diluting a cell culture comprising the pathogen; and
determining in said serial dilutions time to a predetermined OD, the time required for each of said serial dilutions to gain a predetermined OD correlates to its respective colony count.

26. A method of diagnosing an infection in a subject, the method comprising:
(a) serially diluting a cell culture comprising a fluid sample of the subject; and
(b) determining in said serial dilutions time to a predetermined OD, the time required for each of said serial dilutions to gain a predetermined OD being indicative of the infection.

27. The method of claim 26, wherein said fluid sample is selected from the group consisting of urine, cerebrospinal fluid, semen, vaginal discharge, plasma and blood.

28. The method of claim 26, further comprising confirming a result of the diagnosis.

29. The method of claim 26, further comprising informing the subject of a result of the diagnosis.

30. The method of claim 26, wherein said cell culture comprises culture medium.

31. The method of claim 26, wherein the infection is a bacterial infection or a fungal infection.

32. (canceled)

33. A method of determining clonality of a cell culture, the method comprising:
culturing said cell culture; and
monitoring time to a predetermined OD, wherein a time to OD of a predetermined value is indicative of a monoclonal cell culture.

34. The method of claim 33, further comprising generating a calibration curve of time to OD as a function of CFU.

35. The method of claim 33, wherein said monitoring comprises real-time monitoring.

36. The method of claim 33, further comprising diluting said cell-culture to a single cell culture following said monitoring.

37. The method of claim 33, further comprising testing synchronization of said cell culture.

38. (canceled)

39. The method of claim 9, wherein said cell culture comprises transformed cells.

40. A method of determining clonality of a cell culture, comprising analyzing the culture according to the method of claim 9.

41. A method of cloning comprising:
transforming competent cells with a polynucleotide of interest, so as to obtain transformed cells;
identifying a clone expressing said polynucleotide of interest according to the method of claim 9;
sequencing said clone so as to identify said polynucleotide of interest.

42. The method of claim 9, being automated.

43. (canceled)

44. The A method of synchronizing a plurality of cell cultures, the method comprising:
simultaneously monitoring OD of each of said plurality of cell cultures, wherein an OD distribution range which exceeds a predetermined value is indicative of non-synchronized cell cultures; and
diluting said cell cultures of said plurality of cell cultures exhibiting an OD value which exceeds a predetermined value so as to minimize said OD distribution range and synchronize said plurality of cell cultures.

45-48. (canceled)
